(19) United States
(12) Patent Application Publication
(10) Pub. No.: US 2004/0220127 A1
(43) Pub. Date: Nov. 4, 2004

(54) Methods and compositions relating to 5'-chimeric ribonucleic acids

(76) Inventors: Paul Sternberg, Pasadena, CA (US); Byung Joon Hwang, San Marino, CA (US)

Correspondence Address: ROPES & GRAY LLP, ONE INTERNATIONAL PLACE, BOSTON, MA, 02110-2624, US

(21) Appl. No.: 10/639,016

(22) Filed: Aug. 11, 2003

Related U.S. Application Data
(60) Provisional application No. 60/423,490, filed on Nov. 4, 2002.
(60) Provisional application No. 60/402,473, filed on Aug. 9, 2002.

(51) Int. Cl. A61K 48/00; C12Q 1/68; C12P 19/34

(52) U.S. Cl. 514/44; 435/6; 435/91.2

(57) ABSTRACT

The disclosure provides, among other things, methods for producing and using 5′-chimeric RNAs and cDNAs. 5′-chimeric RNAs and cDNAs may be used, for example, for high-throughput analysis of the 5′-end sequences for RNA transcripts.

### RELATED APPLICATION

This application claims the benefit of the filing date of U.S. Provisional Application No. 60/402,473, filed Aug. 9, 2002, entitled “Genome-wide scanning 5′end of mRNA transcripts using a new technique ‘trans-splicing coupled serial analysis of gene expression (TSC-SAGE)’”, by P. W. Sternberg and B. J. Hwang, and U.S. Provisional Application No. 60/423,490, filed Nov. 4, 2002, entitled “Methods and compositions relating to 5′-chimeric ribonucleic acids”, by P. Sternberg and B. Hwang. The entire teachings of the referenced applications are incorporated by reference herein.

### BACKGROUND

With the completion of whole genome sequences for humans and many commonly-used experimental organisms, the biomedical research community has invested significant resources in an effort to identify and catalog all of the genes and ribonucleic acid (RNA) transcripts encoded within these genomes. Genomic DNA sequence information is enzymatically read, or transcribed, to produce RNA transcripts. These transcripts direct the production of proteins that perform most of the biochemical functions of an organism, although certain RNA transcripts also perform significant biochemical functions. Transcripts that encode polypeptides are generally referred to as messenger RNAs (mRNAs). Other kinds of transcripts include ribosomal RNAs (rRNAs) and translation RNAs (tRNAs). An RNA is generally a linear polymer; one end of the polymer is termed the 5′ end, and the other end is termed the 3′ end. RNAs are synthesized during transcription in a 5′ to 3′ direction, and any polypeptide encoded by an mRNA is encoded with a 5′ to 3′ directionality. Accordingly, the 5′ end of an RNA is generally referred to as the beginning, or the “upstream” end of the transcript. Most mRNAs in eukaryotes, and some other RNAs, have a polyadenylated tail at the 3′ end (a “poly-A tail”). These RNAs are often referred to as poly-A RNAs.

The process of identifying the complete set of transcripts produced by an organism has proven to be technically difficult and has become a rate-limiting step in the process of understanding the nature of genome organization. Our limited understanding about complexity and diversity of the completed genomes prevents the correct computer-based prediction of genes and RNA transcripts within genome sequence. Furthermore, it has been observed that present methods for predicting gene sequences in genomes underestimate the number of different transcripts produced by cells. Alternative transcription initiation in a single gene, along with alternative RNA splicing and alternative transcription termination, may be responsible for this higher transcript diversity. For example, the number of predicted genes in the human genome sequence is lower than expected, based on the relatively large size of the human genome. The ratio of predicted genes to genome size is smaller in humans than in other eukaryotes of apparently lower complexity, such as Caenorhabditis elegans and Drosophila melanogaster. However transcript diversity in humans may be substantially higher than the number of predicted genes. The draft of the human genome sequence predicted that at least 50% of the human genes have alternative RNA transcripts, suggesting that large-scale research to identify alternative RNA transcripts will contribute significantly to our understanding of human biology.

The most commonly used experimental method for identifying genes and RNA transcripts is a technique called expressed sequence tag (EST) analysis. EST analysis generally involves randomly synthesizing complementary DNA (cDNA) from RNA transcripts, and sequencing the resulting cDNAs to obtain the sequence for a portion of the transcript (the expressed sequence tag). EST analysis is biased towards the identification of the 3′-end of transcripts, meaning that an EST survey of an organism generally identifies sequences from the middle or end portions of RNAs. EST analysis often fails to detect rare transcripts, and EST analysis is of limited utility for identifying the true 5′-end of an RNA transcript. In particular, when different RNA transcripts are produced from a single gene by alternative transcription initiations, EST sequencing will not usually distinguish the shorter full-length transcripts from partially degraded versions of the longer transcripts. EST analysis also requires tedious sequencing of each individual cDNA that is isolated.

Accordingly, improved methods for the analysis of RNA transcripts would be useful for a variety of purposes, including accelerating the extraction of information from genomic sequences.

### BRIEF SUMMARY

In certain aspects, the disclosure provides methods for producing RNAs and cDNAs that have an additional sequence added at or near the 5′-end, termed “5′-chimeric RNAs” and “5′-chimeric cDNAs”. In certain aspects, the disclosure provides methods for using 5′-chimeric RNAs and cDNAs to obtain information, and particularly sequence information, about the 5′-regions of transcripts. In certain aspects, the disclosure provides methods for using 5′-chimeric RNAs and cDNAs to obtain information about both the 5′- and 3′-regions of transcripts. Information about the 5′-regions of transcripts, and optionally the 3′-regions, may be used for a variety of purposes including, for example, mapping transcription start sites in genomes, identifying novel transcription start sites, identifying novel transcripts, identifying rare transcripts, characterizing global gene and protein expression in specific cell types, and preparing probe arrays that hybridize to sequences from the 5′-regions of transcripts. In certain aspects, the methods provided herein are suitable for high-throughput analysis and may be used for analysis at the genomic scale. In certain aspects, methods provided herein are suitable for genome-scale analysis of organisms for which a genome sequence has not yet been obtained. In additional aspects, the disclosure provides various technologies that, while useful in the analysis of 5′- and 3′-regions of transcripts, may be used independently for a variety of purposes. In certain aspects the disclosure provides compositions of matter and kits that relate to the methods described herein.

In certain aspects, the disclosure provides 5′-trans-splicing nucleic acids for affixing an exon sequence to the 5′-regions of transcripts (referred to as “acceptor RNAs” or, more specifically, “splice acceptor RNAs”) in vivo. 5′-trans-splicing nucleic acids may be used, for example, to generate 5′-chimeric nucleic acids. In certain embodiments, a 5′-trans-splicing nucleic acid comprises an exon and an intron, arranged so that the exon is upstream of (5′ relative to) the intron. In general, when a 5′-trans-splicing nucleic acid is expressed as an RNA in a cell, the exon sequence is transferred, via a trans-splicing reaction, to the 5′ portion of an acceptor RNA, making a 5′-chimeric RNA. The junction between the portion of the RNA corresponding to the trans-spliced exon and the portion corresponding to the acceptor RNA is termed the “chimeric junction”. If the exon of the 5′-trans-splicing nucleic acid has a known sequence, then the 5′-chimeric RNA will have a known sequence at or near the 5′-end that may be used to facilitate 5′-RNA end determination (5′-RED) (or 3′ end identification by, e.g., circularization methods) and other methods.

In certain embodiments, a 5′-trans-splicing nucleic acid is based on a naturally occurring trans-splicing nucleic acid, such as the SL-1 or SL-2 splice leader sequences of the nematode Caenorhabditis elegans. For example, the exon of a 5′-trans-splicing nucleic acid comprises a sequence that is at least 80% identical to a sequence selected from the group consisting of SEQ ID NOs: 1, 4, 7 and 8 and optionally at least 85%, 90%, 95%, 99% or 100% identical to a sequence selected from the group consisting of SEQ ID NOs: 1, 4, 7 and 8. In certain embodiments, the intron of a 5′-trans-splicing nucleic acid comprises a sequence that is at least 80% identical to a sequence selected from the group consisting of SEQ ID NOs: 2 and 5, and optionally at least 85%, 90%, 95%, 99% or 100% identical to a sequence selected from the group consisting of SEQ ID NOs: 2 and 5. In certain embodiments, the disclosure provides a 5′-trans-splicing nucleic acid comprising an exon and an intron, wherein the exon comprises a label sequence, such as a sequence motif for recognition by an enzyme or other reagent that binds, modifies or cleaves nucleic acids. A 5′-trans-splicing nucleic acid may be an RNA that is competent to participate in a trans-splicing reaction, and a 5′-trans-splicing nucleic acid may also be nucleic acid, such as a DNA, having a sequence that is the sense or antisense of the RNA. In certain embodiments, a 5′-trans-splicing nucleic acid is a double-stranded DNA for expression of an RNA that is competent to participate in a trans-splicing reaction.

In certain aspects, the disclosure provides methods for producing 5′-chimeric RNAs by expressing a 5′-trans-splicing nucleic acid in a cell. In certain embodiments, an RNA preparation is obtained from the cell, and the RNA preparation comprises splice acceptor RNAs that have the exon portion of the 5′-trans-splicing nucleic acid affixed at or near the 5′-ends. While not wishing to be bound to theory, it is generally expected that the exon of a 5′-trans-splicing RNA is spliced onto the acceptor RNA (e.g. an mRNA) at a position having the characteristics of a 3′-splice site (e.g. an A-G sequence), with sequence upstream of the 3′-splice site in the acceptor RNA being eliminated in the splicing reaction. Accordingly, it is expected that the junction between the added exon and the acceptor RNA will not usually correspond to the exact 5′-end of the acceptor RNA. In certain embodiments, the disclosure provides methods for producing 5′-labeled chimeric RNAs by expressing a 5′-trans-splicing nucleic acid in a cell, wherein the 5′-trans-splicing nucleic acid comprises an intron and an exon, and wherein the exon comprises a label sequence.

In certain aspects, the disclosure provides methods for enriching for RNA molecules comprising an exon of a 5′-trans-splicing nucleic acid. In certain embodiments, an RNA preparation is enriched for RNA molecules comprising the exon of the 5′-trans-splicing nucleic acid by a method comprising: (1) contacting the RNA preparation with an exon purification oligonucleotide having a sequence that is complementary to at least a portion of the exon, and (2) discarding RNA molecules that do not bind to the exon purification oligonucleotide.

In certain aspects, the disclosure provides methods for depleting RNA molecules comprising an intron of a 5′-trans-splicing nucleic acid from an RNA preparation. Methods of this type are useful, for example, for separating 5-chimeric RNAs from 5′-trans-splicing RNAs that have not participated in a trans-splicing reaction. In certain embodiments, an RNA preparation is depleted for RNA species comprising the intron of the 5′-trans-splicing nucleic acid by a method comprising (1) contacting the RNA preparation with an intron purification oligonucleotide having a sequence that is complementary to at least a portion of the intron, and (2) discarding RNA molecules that do bind to the intron purification oligonucleotide.

In certain aspects, the disclosure provides methods for producing a 5′-labeled chimeric cDNA. In certain embodiments, a 5′-labeled chimeric cDNA is prepared by a method comprising: (1) obtaining an RNA preparation from a cell expressing a 5′-trans-splicing nucleic acid; (2) synthesizing an antisense cDNA strand; and (3) synthesizing a sense cDNA strand using an oligonucleotide primer comprising a label, optionally a label sequence, an attached label and/or an incorporated label. In certain embodiments, a 5′-labeled chimeric cDNA is prepared by a method comprising: (1) obtaining an RNA preparation from a cell expressing a 5′-trans-splicing nucleic acid (2) synthesizing an antisense cDNA by incubating the RNA preparation, or a portion thereof, in a mixture comprising a downstream primer and a reverse transcriptase; (3) synthesizing a double-stranded cDNA by incubating an antisense cDNA in a mixture comprising an upstream primer that hybridizes to at least a portion of the exon of the 5′-trans-splicing nucleic acid and an enzyme that mediates sense-strand synthesis; and (4) synthesizing a 5′-labeled chimeric cDNA by incubating a double-stranded cDNA, or copy thereof, in a mixture comprising an upstream primer that comprises a label, a downstream primer and an enzyme that mediates polynucleotide synthesis. Optionally additional cDNA copies are synthesized before or after using a labeled oligonucleotide primer. Optionally the label is a label sequence, an attached label and/or an incorporated label. In certain embodiments a 5′-labeled chimeric cDNA is produced by synthesizing a cDNA based on a 5′-labeled chimeric RNA produced in a cell expressing a 5′-trans-splicing nucleic acid comprising an exon having a label sequence.

In certain embodiments a 5′-labeled chimeric cDNA may be produced by forming a polynucleotide synthesis mixture that comprises: (1) a cDNA preparation derived from a cell population expressing a 5′-trans-splicing nucleic acid; (2) a primer that has a sequence that hybridizes to at least a portion of the exon and wherein the primer comprises a label; and (3) an enzyme that catalyzes polynucleotide synthesis. The mixture is incubated under conditions that permit polynucleotide synthesis, thereby producing a 5′-chimeric cDNA comprising a label at or near the 5′ end of the sense strand (a 5′-labeled chimeric cDNA). In certain embodiments a primer for use in synthesizing a cDNA comprises a label, such as an attached label, an incorporated label and/or or a label sequence. In certain embodiments the attached label and/or incorporated label is a label that facilitates detection and/or purification of the primer and the cDNA that the primer is incorporated into. In certain embodiments, the label is a sequence motif that is a recognition site for an enzyme or reagent that binds, cleaves or modifies a nucleic acid, such as a restriction enzyme. In certain embodiments, the primer is designed to introduce a sequence change into the cDNA. For example, a primer may introduce a label sequence that was not originally present in the 5′-chimeric cDNA. In certain embodiments, a label sequence is already present in the 5′-chimerc cDNA (e.g. the label sequence may be introduced in vivo as part of the trans-spliced exon sequence), and oligonucleotide serves to copy the sequence motif.

In certain embodiments, a 5′-labeled chimeric cDNA prepared according to a method disclosed herein comprises a sequence motif for recognition by an enzyme or reagent that binds, cleaves or modifies nucleic acids. In certain embodiments, 5′-labeled chimeric cDNA comprises a cleavage reagent recognition site, preferably located at or near the 3′-end of the portion of the cDNA that corresponds to an exon added to an RNA by a trans-splicing reaction with a 5′-trans-splicing nucleic acid. In certain embodiments, the cleavage reagent cleaves, or permits cleavage, at a site external to the recognition site. In certain embodiments, the cleavage reagent is a restriction enzyme, and preferably the restriction enzyme is selected from the group consisting of: a type IIs restriction enzyme and a type III restriction enzyme.

In further aspects, the disclosure provides in vitro methods for producing 5′-labeled chimeric RNA and cDNA. In certain embodiments, an in vitro method comprises selectively ligating an oligonucleotide to the 5′ end of an RNA (the acceptor RNA), preferably a full-length or capped mRNA. Optionally the oligonucleotide is an single-stranded RNA, a single-stranded DNA, a single-stranded RNA-DNA hybrid, or a duplex combining any of the preceding. In certain preferred embodiments, selectively ligating an oligonucleotide to the 5′-end of a capped RNA in an RNA preparation comprises exposing the RNA preparation to an enzyme that catalyzes the removal of phosphate from the 5′ end of RNA molecules not having a 5′-cap, then using an enzyme that catalyzes the conversion of an RNA comprising a 5′-cap into an RNA comprising a 5′-phosphate and then using a ligase to ligate the oligonucleotide to the RNA having a free 5′-phosphate. In certain embodiments, the enzyme that catalyzes the removal of phosphate from the 5′ end of RNA molecules not having a 5′-cap is calf intestinal phosphatase. In certain embodiments, the enzyme that catalyzes the conversion of an RNA comprising a 5′-cap into an RNA comprising a 5′-phosphate is tobacco acid pyrophosphatase. In certain embodiments the ligase is a T4 RNA ligase. In certain embodiments, the oligonucleotide comprises a label, such as an attached label, incorporated label and/or a label sequence. In certain embodiments, the attached label or incorporated label is a label for purification and/or detection of the oligonucleotide and the RNA to which it is ligated. In certain embodiments the label sequence is a recognition site for an enzyme or reagent that binds, cleaves or modifies nucleic acids, such as a restriction enzyme. In certain embodiments, cDNA is synthesized from the ligated RNA to give 5′-chimeric cDNA. The junction between the sequence corresponding to the added oligonucleotide and the acceptor RNA is termed the “chimeric junction”.

In certain aspects, the disclosure provides methods for identifying sequences at or near the 5′ ends of cDNAs. In certain embodiments, a method disclosed herein comprises: (1) digesting 5′-chimeric cDNAs with a tagging cleavage reagent that cleaves at a position external to the recognition site in the 3′ direction, thereby releasing 5′ portions of the chimeric cDNAs and 3′ portions of the chimeric cDNAs; (2) selectively obtaining the 5′ portions; and (3) sequencing a plurality of the 5′ portions of the cDNA. In certain embodiments, a plurality of the 5′-chimeric cDNAs comprise an affinity purification label at or near their 5′-ends, and optionally, selectively obtaining the 5′ portions of the chimeric cDNAs comprises contacting the digested mixture with a capture medium that binds to the affinity purification label. Optionally the tagging cleavage reagent cleaves at a position at least 7, 10 or 14 base pairs distant in the 3′ direction from the 3′-end of the recognition site. In certain embodiments, sequencing a plurality of the 5′ portions of the chimeric cDNAs involves selectively sequencing that portion of the cDNAs that is immediately downstream of the chimeric junction. In certain embodiments, sequencing a plurality of the 5′ portions of the chimeric cDNAs comprises forming one or more nucleic acid concatemers comprising a plurality of 5′ portions of the cDNAs and sequencing one or more of the concatemers. In certain embodiments, nucleic acid concatemers are formed by a method comprising: (1) ligating a adapter to the 3′ ends of the selectively obtained 5′ portions of the digested chimeric cDNAs to produce cDNA-adapter constructs; (2) amplifying the cDNA-adapter constructs using a 5′ oligonucleotide primer comprising a first anchor cleavage reagent recognition site and a 3′ oligonucleotide primer comprising a second anchor cleavage reagent recognition site, thereby obtaining amplified products comprising flanking first and second anchor cleavage reagent recognition sites; (3) digesting the amplified products with the first and second anchor cleavage reagents to obtain double-digested amplified products; and (4) ligating the double-digested amplified products together to form nucleic acid concatemers. In certain embodiments, methods for identifying sequences at or near the 5′ ends of chimeric cDNAs employ 5′-chimeric cDNAs derived from 5′-chimeric RNAs that result from a trans-splicing reaction between a 5′-trans-splicing nucleic acid and an splice acceptor RNA. In further embodiments, methods for identifying sequences at or near the 5′ ends of chimeric cDNAs employ 5′-chimeric cDNAs derived from 5′-chimeric RNAs prepared by selectively ligating an RNA oligonucleotide to the 5′-end of capped RNAs. In certain embodiments, a cDNA comprising between 7 and 50 nucleotides of sequence that corresponds to sequence at or near the 5′-end of an acceptor RNA is termed a “TAG” or “5′-TAG” and the sequence information obtained from sequencing a TAG is termed a “TAG sequence”. In certain embodiments, a preferred TAG comprises between 14 and 30 nucleotides of sequence that corresponds to a sequence at or near the 5′-end of an acceptor RNA.

In certain embodiments, the disclosure provides suppression subtractive hybridization—polymerase chain reaction (SSH-PCR) methods for decreasing the differences in the abundance of or representation of RNA or cDNA species, as well as fragments thereof. In certain embodiments, the disclosure provides methods for normalizing the amount of cDNA species, or 5′ cleavage fragments thereof, the method comprising performing suppression subtractive hybridization-polymerase chain reaction (SSH-PCR) using a driver consisting essentially of a 5′ cleavage fragment. A 5′ cleavage fragment is released from a cDNA by digestion with a cleavage reagent and corresponds to a portion at or near the 5′-end of the cDNA. In certain embodiments, a 5′ cleavage fragment driver is hybridized to a first tester and a second tester, wherein the first tester comprises a 5′-cleavage fragment, and wherein the second tester comprises a 5′-cleavage fragment. In certain embodiments, the disclosure provides methods comprising: (a) preparing a population of nested 5′ cleavage fragments derived from a population of 5′-labeled chimeric cDNAs, wherein a nested 5′ cleavage fragment comprises a sequence corresponding to a sequence of an acceptor RNA and a 5′-flanking region and/or a 3′-flanking region; (b) preparing a population of free 5′ cleavage fragments derived from the population of 5′-labeled chimeric cDNAs; (c) forming a mixture of a population of denatured nested 5′ cleavage fragments and a population of denatured free 5′ cleavage fragments and allowing renaturation; (d) selectively amplifying nested 5′ cleavage fragments that are not hybridized to free 5′ cleavage fragments. In certain embodiments, the disclosure provides SSH-PCR methods that normalize the abundance or representation of alternative transcripts encoded by the same gene but having distinct 5′-ends. In certain embodiments, the disclosure provides methods for identifying rare alternative transcripts.

In certain aspects the disclosure provides methods for obtaining sequence from at or near both the 5′- and 3′-ends of an RNA. In certain embodiments, the disclosure provides a method for making a 5′-3′-labeled chimeric cDNA, comprising: (a) selectively removing at least a portion of the 3′ poly-A region of a 5′-labeled chimeric cDNA, wherein the 5′-labeled chimeric cDNA comprises a recognition site for a first tagging cleavage reagent; and (b) ligating a 3′-adaptor to the 3′-end of the 5′-labeled chimeric cDNA to make a 5′-3′-labeled chimeric cDNA, wherein the 3′-adaptor comprises a recognition site for a second tagging cleavage reagent. The 5′-3′-labeled chimeric cDNA may be circularized by, for example, intramolecular ligation. In certain embodiments the disclosure provides methods for making 5′-3′-TAGs, the methods comprising providing a circularized 5′-3′-labeled chimeric cDNA, where the 5′-3′-labeled chimeric cDNA comprises: (i) a sequence corresponding to an acceptor RNA; (ii) a 5′-chimeric sequence attached to the 5′-end, including a recognition site for a first tagging cleavage reagent; and (iii) a 3′-adaptor sequence attached to the 3′-end, including a recognition site for a second tagging cleavage reagent. Since the cDNA is circularized, the 3′-end of the 3′-adaptor is attached to the 5′-end of the 5′-chimeric sequence. The circularized 5′-3′-labeled chimeric cDNA is digested with the first and second cleavage reagents (which may be the same single cleavage reagent) to release a linearized, double-digested product; and the product is circularized to give a product comprising, in order: the 5′-chimeric sequence, a 5′-TAG sequence corresponding to a 5′-portion of an acceptor RNA, a 3′-TAG sequence corresponding to a 3′portion of the acceptor RNA and the 3′-adaptor sequence. A 5′-3′-TAG is made by amplifying the circularized product using 5′-primer that hybridizes to the 5′-chimeric sequence and comprises a recognition site for a first anchoring cleavage reagent and a 3′-primer that hybridizes to the 3′-acceptor sequence and comprises a recognition site for a second anchoring cleavage reagent, thereby obtaining an amplified product, and then digesting the amplified product with the first and second anchoring cleavage reagent to release a 5′-3′-TAG. The 5′-3′-TAGs may be cloned and sequenced or concatemerized, cloned and then sequenced as concatemers.

In certain aspects, the disclosure provides methods and computer-assisted methods for identifying a TAG sequence within a genomic sequence, for identifying a 5′-end of a transcript corresponding to a TAG sequence for assessing various characteristics of a transcript corresponding to a TAG sequence. In certain aspects, a TAG sequence may be compared to one or more nucleotide sequence databases, including genomic sequence databases, cDNA databases and EST databases. In certain embodiments the TAG sequence is derived from a 5′-chimeric cDNA or 5′-3′-chimeric cDNA. In certain embodiments, the TAG sequence is compared to a genomic sequence. In certain embodiments, it may be determined whether the genomic sequence corresponding to the TAG encodes a transcript having the same 5′-3′ directionality as the TAG sequence. In certain embodiments where the TAG sequence is derived from a trans-splicing based methodology, it may be determined whether the TAG sequence is located after a splicing acceptor site (e.g. an A-G sequence). In certain embodiments, if the TAG sequence is identified at more than one position in a genomic sequence, the position where the TAG sequence follows a splice acceptor sequence may be considered the correct TAG sequence position. When a TAG matches a position in a genomic sequence, the 5′-end of the transcript containing the TAG may be inferred using one or more distance parameters. Optionally, a distance parameter is the distance from the TAG sequence in the genome to the first nearby exon. Optionally, a distance parameter is the distance from the TAG sequence in the genome to the nearest upstream initiator (e.g. ATG) codon for the gene. Optionally, both of the preceding distance parameters are employed. In certain embodiments, one or more distance parameters are used to determine whether the 5′-end of a transcript corresponding to a TAG is a new gene, an alternative transcript of a known gene or a known transcript of a known gene. One or more analyses of a TAG sequence may be performed on a computer, and instructions for analyzing a TAG sequence may be entered into a computer and/or onto a computer-readable storage medium, such as an optical or magnetic storage medium.

In certain aspects, the disclosure provides methods for identifying, in a genome of a reference organism, a genomic region that is likely to encode a transcript that is an ortholog of a transcript of a test organism. Optionally the test organism is an organism for which there is incomplete or insignificant genomic sequence available. Generally the reference organism is one for which a significant amount of genomic sequence is available, and preferably a complete or near-complete genome sequence is available for the reference organism. In certain embodiments, the disclosure provides a method comprises accessing a 5′-TAG sequence and a 3′-TAG sequence derived from the transcript of the test organism; identifying in the genome of the reference organism one or more sequences that match or closely match the 5′ TAG sequence and the 3′ TAG sequence to obtain one or more 5′- and 3′-orthologous genomic sequences. Optionally, an orthologous genomic sequence shares 80% or greater sequence identity with the corresponding TAG sequence, and preferably 85%, 90%, 95% or greater sequence identity. In particularly preferred embodiments, the orthologous genomic sequence is 100% identical to the corresponding TAG sequence. A genomic region comprising a 5′-orthologous genomic sequence and a 3′-orthologous sequence in an appropriate orientation is a genomic region that is likely to encode a transcript corresponding to the transcript of the test organism. By appropriate orientation is meant that the 5′- and 3′-orthologous sequences are oriented in the genome with respect to each other in a manner that is consistent with both sequences being transcribed as part of a single transcript.

In further aspects, the disclosure provides methods for producing labeled fusion proteins. In certain embodiments, the method comprises expressing in a cell a 5′-trans-splicing nucleic acid that comprises an exon encoding a polypeptide label. The exon is transferred to one or more splice acceptor RNA molecules in the cell, creating 5′-chimeric RNAs encoding fusion proteins that comprise the polypeptide label at the amino-terminus. In certain embodiments, the polypeptide label is a purification label, and optionally the fusion proteins are purified or partially purified using the purification label. In certain embodiments, the polypeptide label is a detection label. Optionally, the fusion proteins are detected in vivo by detecting the label, and optionally the fusion proteins are detected in vitro by preparing a cell fraction or polypeptide composition from the cell and detecting the detection label in the cell fraction or polypeptide composition. In certain embodiments, labeled fusion proteins are produced in specific cell types, and optionally used for cell-specific proteomic analysis.

In certain aspects, the disclosure provides oligonucleotides for use in generating 5′-labeled cDNAs. In certain embodiments, the disclosure provides an oligonucleotide of between 15 and 100 nucleotides in length, comprising an attached or incorporated label and a recognition site for a cleavage reagent that cleaves DNA at a position external to the recognition site, and preferably in the 3′ direction. In certain embodiments, the cleavage reagent is a restriction enzyme that cleaves DNA at least 7, 10 or 14 base pairs distant from the recognition site, preferably in the 3′ direction. In certain embodiments, the recognition site is positioned within the 10 base pairs closest to the 3′-end of the oligonucleotide, and preferably the recognition site is positioned at the 3′-end of the oligonucleotide primer. In certain embodiments, the attached or incorporated label is selected from the group consisting of: an affinity purification label and a fluorophore.

In certain aspects, the disclosure provides modified SL-1 5′-trans-splicing nucleic acids. In certain embodiments, the disclosure provides 5′-trans-splicing nucleic acids that comprise an exon sequence at least 80%, 85%, 90%, 95%, 99% or 100% identical to a sequence selected from the group consisting of SEQ ID NOs:7 and 8.

In certain aspects, the disclosure provides nucleic acid concatemers comprising a plurality of TAGs corresponding to transcripts. In certain embodiments, a nucleic acid concatemer comprises one or more iterated units, wherein each iterated unit comprises, in order, a first TAG, a first cleavage reagent recognition site, a second TAG and a second cleavage reagent recognition site, wherein a TAG is a nucleic acid sequence of between 7 and 50 base pairs in length that corresponds to at least one transcript. A concatemer may be generated by novel methods, disclosed herein, that employ a plurality of adapter types, each comprising a different anchoring cleavage reagent recognition site. The disclosure further provides concatemers generated by such a method, wherein the concatemers comprise cleavage reagent recognition sites between TAG sequences, and wherein the concatemers comprise recognition sites for three or more different cleavage reagents. In certain embodiments, forming nucleic acid concatemers comprises: i) ligating one of n different adapters to the 3′ end of the selectively obtained 5′ portions of cDNA, wherein each of the n different adapters comprises a distinct second anchor cleavage reagent recognition site or the absence of a second anchor cleavage reagent recognition site, thereby making n populations of cDNA-adapters having a common first anchor cleavage reagent recognition site at or near the 5′ end having a distinct second anchor cleavage reagent recognition site or no second anchor cleavage reagent recognition site at or near the 3′ end, with the proviso that no more than two of the n different adapters have no second anchor cleavage reagent recognition site; ii) forming nucleic acid concatemers by the iterated process of digestion with each distinct second anchor cleavage reagent and directed ligation of the digested nucleic acid ends. Optionally, n is six, and the first adapter comprises a first second anchor cleavage reagent recognition site, the second adapter comprises a second second anchor cleavage reagent recognition site, the third adapter comprises a third second anchor cleavage reagent recognition site, the fourth adapter comprises a fourth second anchor cleavage reagent recognition site, and the fifth and sixth adapters do not have a second anchor cleavage reagent recognition site.

In certain aspects, the disclosure provides 5′-3′-TAGs, comprising a 5′-TAG sequence corresponding to sequence at or near the 5′-end of an RNA and a 3′-TAG sequence corresponding to the sequence at or near the 3′-end of the same RNA, and preferably at or near the 3′-end of the portion of the RNA that is encoded by the genome (as opposed to post-transcriptionally added sequence, such as portions of the poly-A tail). Preferably, the 5′-TAG and the 3′-TAG are positioned in opposite orientation, and in particularly preferred embodiments, the 5′-TAG and 3′-TAG are positioned with their 3′-ends proximal to each other and their 5′-ends distal to each other (i.e. tail-to-tail orientation). In certain embodiments, the disclosure provides concatemers of 5′-3′-TAGs, comprising one or more iterated units, wherein an iterated unit comprises, in order, a first cleavage reagent recognition site, a first 5′-3′-TAG, a second cleavage reagent recognition site and a second 5′-3′-TAG.

In certain aspects, the disclosure provides nucleic acid constructs for the expression of 5′-trans-splicing nucleic acids, comprising a 5′-trans-splicing nucleic acid operably linked to a promoter. In certain embodiments, the disclosure provides a construct comprising (1) a 5′-trans-splicing nucleic acid having an intron and an exon that is positioned 5′ relative to the intron; and (2) a promoter that stimulates expression of the 5′-trans-splicing nucleic acid in a cell. In certain preferred embodiments, the promoter stimulates expression in a cell selected from the group consisting of: a chordate cell, a protozoan cell, an arthropod cell, a fungal cell, a plant cell, a nematode cell and a trematode cell. Optionally, the promoter is a constitutive promoter, a cell-specific promoter or a conditional promoter. Optionally the nucleic acid construct is situated in a vector and/or in a chromosome of a cell.

In certain embodiments, the disclosure provides cells comprising a 5′-trans-splicing nucleic acid, nucleic acid construct or vector disclosed herein. In certain embodiments, the 5′-trans-splicing nucleic acid, nucleic acid construct or vector is integrated into a chromosome. In certain embodiments, the 5′-trans-splicing nucleic acid, nucleic acid construct or vector is present as an episome. In certain embodiments, the cell is selected from the group consisting of: a chordate cell, a protozoan, an arthropod, a fungus, a plant, a nematode and a trematode. In certain embodiments, the cell is a bacterial cell, such as an Escherichia coli cell, preferably used for maintenance or propagation of a vector.

In certain aspects, the disclosure provides probe arrays, such as microarrays or macroarrays, based on sequences at or near the 5′-ends of transcripts. In certain embodiments, the disclosure provides probe arrays comprising a plurality of probes having sequences that correspond to sequences at or near the 5′-ends of a plurality of RNAs. An array will typically have probes positioned at defined spatial positions (a “spatially addressable array”). Optionally, an array will comprise at least 100, at least 500, at least 1000, at least 5000 or at least 10,000 different probes having sequences that correspond to sequences at or near the 5′-ends of a plurality of RNAs. In reference to probe arrays, a sequence is “near” the 5′-end of an RNA if it is close enough to the 5′-end to distinguish between alternative transcripts having different 5′-ends. In certain embodiments, the disclosure provides probe arrays comprising probes that distinguish between 5′ alternative transcripts (transcripts encoded by the same gene but having different 5′-ends) encoded by a plurality of genes. Optionally, a probe array is able to distinguish between two or more 5′-alternative transcripts for at least 100 genes, at least 500 genes, at least 1000 genes, at least 5000 genes or at least 10,000 genes. In certain embodiments, the disclosure provides methods for making probe arrays. In certain embodiments, a probe array may be made by identifying sequences at or near the 5′-end of a plurality of RNAs, making probes based on the sequences at or near the 5′-ends and affixing the probes to a spatially addressed array.

In certain aspects, the disclosure provides kits for use in generating 5′-chimeric RNAs. In certain embodiments, the disclosure provides kits comprising: a vector comprising a 5′-trans-splicing nucleic acid operably linked to a promoter and a single-stranded oligonucleotide that hybridizes to at least a portion of the exon of the 5′-trans-splicing nucleic acid. Preferably the single stranded oligonucleotide comprises an attached label, an incorporated label and/or a label sequence. In certain embodiments, the label sequence is a recognition site for a cleavage reagent that cleaves DNA at a position at least 7 base pairs distant from the recognition site, and the recognition site is positioned within the 10 base pairs closest to the 3′ end of the oligonucleotide primer. In certain embodiments, the attached or incorporated label is an affinity purification label. In certain aspects, the disclosure provides kits comprising: an oligonucleotide for attachment to the 5′-end of an acceptor RNA and a single-stranded oligonucleotide of between 15 and 100 nucleotides in length that hybridizes to at least a portion of the oligonucleotide for attachment to the 5′-end of an acceptor RNA. Preferably the single stranded oligonucleotide comprises an attached label, an incorporated label and/or a label sequence. In certain embodiments, the label sequence is a recognition site for a cleavage reagent that cleaves DNA at a position at least 7 base pairs distant from the recognition site, and the recognition site is positioned within the 10 base pairs closest to the 3′ end of the oligonucleotide primer. In certain embodiments, the attached or incorporated label is an affinity purification label. Optionally, the oligonucleotide for attachment to the 5′-end of an acceptor RNA is single-stranded or double-stranded, and optionally it is RNA, DNA, an RNA/DNA hybrid or double-stranded mixtures thereof.

In certain embodiments, the disclosure provides kits for the analysis of 5′-ends of transcripts. In certain embodiments the disclosure provides kits comprising two or more of the following: a tagging cleavage reagent, a first anchoring cleavage reagent, a second anchoring cleavage reagent, a 3′-linker, a 5′-primer comprising a recognition sequence for the first anchoring cleavage reagent and a 3′-primer comprising a recognition sequence for a second anchoring cleavage reagent.

In certain embodiments, the disclosure provides methods and kits for normalizing the abundance of nucleic acids in a sample comprising nucleic acid species of differing abundances. Such a method may comprise contacting the nucleic acid species with a population of randomized oligonucleotides in conditions that are conducive to specific hybridization between the nucleic acid species and complementary randomized oligonucleotides; and selectively recovering the nucleic acid species hybridized to the random oligonucleotides, wherein the recovered nucleic acid species have a decreased variation in abundance relative to the initial sample. Optionally, the randomized oligonucleotides include an affinity purification label. Optionally, the randomized oligonucleotides are affixed to a substrate. In certain embodiments, selectively recovering the nucleic acid species hybridized to the random oligonucleotides comprises contacting the nucleic acids with a capture medium comprising an agent that binds specifically to the affinity purification label; disrupting the hybridization between the nucleic acid species and the random nucleotides; obtaining the released nucleic acid species. Optionally, the randomized oligonucleotides are longer than or equal to 7 bases in length, or at least 9, 10, 11, 12, 13, 14, 15, or 16 bases in length. In a preferred embodiment the randomized oligonucleotides are 14 bases in length. The nucleic acid species may be single stranded (e.g. RNAs generated by an RNA polymerase from a DNA template, or DNAs generated by asymmetric PCR) or double stranded.

In certain aspects, the disclosure provides kits for use in normalizing the abundance of nucleic acid species in a sample. Such a kit will generally comprise a set of randomized oligonucleotides. Optionally the randomized oligonucleotides comprise an affinity purification label and/or are affixed to a substrate (e.g., beads or a membrane). A kit may comprise oligonucleotides for use in generating single strand copies from double stranded nucleic acids in a sample (e.g., an adapter comprising a T7 RNA polymerase site), and a kit may comprise useful enzymes (e.g. RNA polymerase, DNA polymerase, DNAses, RNAses) as well as useful buffers.

The embodiments and practices of the present invention, other embodiments, and their features and characteristics, will be apparent from the description, figures and claims that follow, with all of the claims hereby being incorporated by this reference into this Summary.

### BRIEF DESCRIPTION OF THE DRAWINGS

FIG. 1: Methods to affix an oligonucleotide at or near the end of a 5′-RNA. A. In vivo and in vitro trans-splicing reactions from a natural splicing leader RNA. B. In vivo and in vitro trans-splicing reactions from a modified splicing leader RNA. C. In vitro RNA-RNA ligation reaction.

FIG. 2: TEC-RED analysis on C. elegans mRNA containing trans-spliced SL-1 exon. Red boxes represent a trans-spliced SL-1 exon. The SL-1 exon sequence is modified during PCR, first to introduce a Bpm I site (step 3), and later to change the Bpm I to an Xho I site during PCR amplification (step 5). Black boxes represent mRNA or cDNA, and blue boxes represent adapter DNA containing a Hae III site. The concatenated TAG polymer (step 7) contains two anchor sequences at both sides of each TAG, which indicate the orientation of each TAG.

FIG. 3: Orientation of TAGs in a concatenated DNA. Xho I/Hae III digestion at step 6 in FIG. 2 releases the TAG fragments containing a small piece of SL-1 exon at the 5′-end and another piece of spliced message cDNA at the 3′-end. These TAGs are directionally concatenated by ligation, forming a TAG polymer as shown here. As the result, 5′-end of each TAG produced by this method is located next to the first anchor restriction enzyme.

FIG. 4: DNA sequencing chromatogram of the TEC-RED analysis. C. elegans mRNA containing SL-1 exon at their 5-ends was subjected to a TEC-RED analysis (FIG. 2). At the last steps in FIG. 2, concatenated TAGs were ligated into plasmid vector for sequencing using a T7 primer. The boxes and annotation indicate the first and second anchor restriction enzyme sites. The anchor sites are located every 14 base pairs (bp).

FIG. 5: Matching TEC-RED data with the genome. (A). Each TAG in every DNA sequencing file is given an ID number. (B). The “AG (splicing acceptor site) plus TAG” sequence is searched within the C. elegans genome sequence. The genomic site matched with the “AG plus TAG” sequence is further analyzed to identify the gene closest to the site. Three different distance (in bp) parameters are calculated from the genomic site matched with the TAG sequence: distance to the first exon, distance to the closest exon, and distance to the nearby ATG of the gene (example in FIG. 6). (C). The TAG sequences are classified by the parameters given in Table 2. (D). Identified new genes are confirmed by reverse transcriptase—polymerase chain reaction (RT-PCR) analysis.

FIG. 6: Splicing acceptor site consensus sequences in C. elegans. Neighboring sequences followed by a splicing acceptor site (AG at −2 and -1 positions) in C. elegans genome are highly conserved. The number inside of parenthesis represents the frequency of each nucleotide at each position.

FIG. 7. An example of searching for C. elegans genome site matching with a TAG sequence. The “AG plus TAG sequence” and its complementary sequence were searched in C. elegans genome sequence, and the search result was processed as described in the text. In this example, TAG identifies an alternative transcript that is transcribed by the second promoter in an intron, because the distance from the TAG sequence to the first exon of this gene is 1601 bp, to the closest exon is 1 bp. The presence of an ATG codon in the TAG sequence (3 bp after the 5′-end of TAG) suggests that a protein can be translated from this transcript.

FIG. 8. In vivo trans-splicing of modified SL-1 (mSL-1) RNA in C. elegans. (A). RT-PCR analysis. The first half of the modified exon sequence of mSL-1 RNA was used as a 5′-PCR primer (labeled as mSL-1). The 3′-PCR primers were specific to each gene (mai-1, gpd-2, and gpd-3). As a control, egl-38 was amplified using its internal primers in all three RNA preparations, suggesting the high and equal quality of RNA preparations. (B). DNA sequencing of the PCR product with mSL-1/gpd-2 primers. A primer from an internal site of gpd-2 was used as sequencing primer.

FIG. 9. Affinity purification of RNA containing mSL-1 exon sequence. Total RNA from C. elegans expressing mSL-1 RNA was applied to an oligonucleotide (complementary to the mSL-1 exon sequence) affinity column. Purified RNA was analyzed by RT-PCR and DNA sequencing.

FIG. 10. Two different methods of processing the ends of TAG molecule. The first method uses a 3′ to 5′ exonuclease activity in T4 DNA polymerase. Treating DNA fragments containing 3′-overhang structure with T4 DNA polymerase removes the overhang structure to blunt their ends. The second method directly ligates the DNA fragments containing the 2 bp-long-3′-overhang structure with an adapter DNA molecules also containing 2 bp-long-3′-overhang structure. Random nucleotides are positioned in the 2 bp-overhang of the adapter DNA. Thus, each adapter molecule can only ligate with the DNA fragment containing the complementary nucleotides.

FIG. 11. Protocol for normalizing 5′-cDNA fragments by single-strand hybridization-polymerase chain reaction (SSH-PCR). (A). Preparation of 5′-cDNA fragments for SSH-PCR. Biotin and Xho I site are introduced to 5′-end of cDNA by PCR using the biotin-primer containing Xho I site. Then, the PCR products are digested with a restriction enzyme, and the 5′-cDNA fragments are purified by streptavidin affinity chromatography. The 5′-cDNA fragments bound onto the column through biotin-streptavidin interaction are ligated with adapter DNA containing Hae III site. After removing the free adapter DNA molecules, the ligated products are divided into three samples and amplified by PCR using different sets of biotin- and non-biotin primers to introduce biotin at the 5′- and/or 3′-end of the PCR products. The 5′-cDNA mixtures are digested with Hae III and/or Xho I and purified by streptavidin affinity chromatography. (B). SSH-PCR of 5′-cDNA fragments. The purified Tester and Driver DNA mixtures are subjected to SSH-PCR to normalize their abundances, following the manufacturer's protocol (CLONTECH). After the subtraction, Xho I site on the trans-spliced exon is changed to Bpm I site by PCR using a mismatched primer, as described in FIG. 2. Now, the PCR products of 5′-cDNA fragments are ready for TEC-RED analysis.

FIG. 12. A scheme to purify mRNA trans-spliced with mSL-1 RNA. The first affinity column contains oligonucleotides complementary to the intron sequence of mSL-1 RNA, and removes unspliced mSL-1 RNA from the RNA mixture (step 1). The unbound RNA is applied to the second affinity column that contains oligonucleotides complementary to the modified exon sequences of mSL-1 RNA and only trans-spliced mRNA is purified by binding to this second column (step 2).

FIG. 13. Linear amplification of full-length cDNA. Green bar sequence, the first half of trans-spliced exon, is used as a primer for reverse transcriptase (step 7) and RT-PCR (step 3). The red bar sequence, the second half of the exon, is used for affinity purification of anti-sense RNA (step 6). The blue bar represents T7 adapter DNA. Several rounds of T7 amplification can be carried out through the multiple cycles of steps 5 to 8. Biotin is attached to the 5′-end of cDNA for affinity purification.

FIG. 14. Selective in vitro RNA-RNA ligation. CIP (Calf Intestine Phosphatase) can not remove the phosphate group from the 5′-end protected by the m7G group. TAP (Tobacco Acid Pyrophosphatase) leaves one phosphate group attached to the 5′-end of RNA. The sequential treatments of these phosphatase, and subsequent ligation with RNA oligonucleotide harboring the recognition site for TAG restriction enzymes, such as Mme I or EcoP15I, selectively attaches the RNA oligonucleotide to the full-length mRNA.

FIG. 15. 5′-LM-RED protocol for human RNA transcripts. The protocol is identical to that of TEC-RED, except the uses of two different restriction enzymes, EcoP15 I (a tagging restriction enzyme) and SnaB I (a second anchor RE). The second EcoP15 site is introduced at the 3′-cDNA end because the enzyme cleaves more efficiently when two recognition sites exist on the same DNA molecule.

FIG. 16. A schematic for analysis of both 5′- and 3′-portions of RNAs (5′-3′-Co-RED), using a circularization method to obtain paired 5′-3′-TAGs.

FIG. 17. The orientation of 5′-3′-TAGs in a genomic sequence as mapped onto a concatemer.

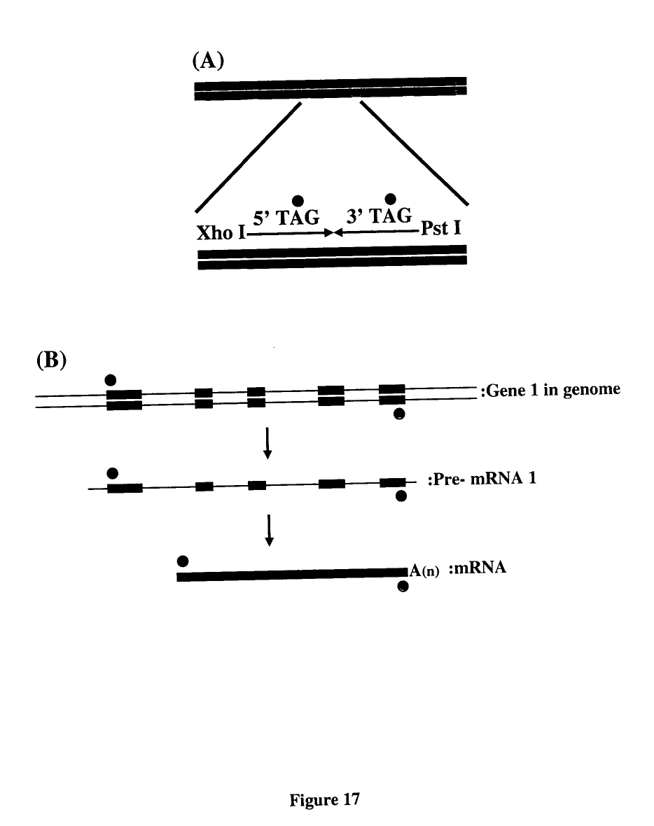

FIG. 18. A schematic for a 5′3′-Co-RED ortholog search using reference genomes.

FIG. 19. A schematic for the efficient concatenation of “TAGs”. In this example, 5′-TAGs released by a cleavage reagent are ligated with four different 3′ adapters, each of which comprises a different restriction enzyme recognition site (second anchor cleavage reagent recognition site). This gives a population of TAGs with a uniform first anchor cleavage reagent recognition site (XhoI in this example) in the 5′ adapter portion, and four different possible second anchor cleavage reagent recognition site in the 3′ adapter portion. Cleavage with the first anchor cleavage reagent, followed by ligation, produces head-to-head (5′-end to 5′-end) ditags, each having a different second anchor cleavage reagent recognition site at each end (depending on how the procedure is performed, there may be a certain percentage of ditags with the same second anchor cleavage reagent recognition site at each end). The ditags are digested with second anchor cleavage reagent number I (RE I) and religated, to form directionally ligated tetra-TAGs. The ditags are then digested sequentially with cleavage reagent numbers 2-4 (RE 2, RE 3, RE 4) with ligation after each digestion. After four rounds of digestion and ligation, the concatemers are composed of 32 TAG units.

FIG. 20. A schematic illustrating a method for obtaining normalized, concatenated 5′ TAGs. The shaded portion shows a normalization step wherein the TAGs are ligated with a 3′ adapter containing a T7 polymerase site. This allows the TAGs to be transcribed into single stranded RNAs. These RNAs are then contacted with a set of immobilized (e.g. on beads) random 14mers. The RNAs that bind to the 14mers are separated and used to regenerate double stranded DNA TAGs, which are then processed, concatenated and sequenced. Since each species of randomized 14mer is present at an equivalent abundance, the abundance of nucleic acids hybridizing to such 14mers will be normalized towards the concentration of the randomized 14mers.

### DETAILED DESCRIPTION

1. Definitions

For convenience, certain terms employed in the specification, examples, and appended claims are collected here. These and other terms are defined and described throughout the disclosure. Unless defined otherwise, all technical and scientific terms used herein have the same meaning as commonly understood by one of ordinary skill in the art to which this invention belongs.

The term “5′-chimeric RNA” refers to an RNA comprising a 3′ portion that corresponds to an acceptor RNA transcript and a 5′ portion (that may be RNA, DNA, etc.) that is trans-spliced, ligated or otherwise affixed at or near the 5′ end of the acceptor RNA transcript. The term “5′-chimeric cDNA” refers to a cDNA comprising sense and/or antisense sequence of a 5′-chimeric RNA. A 5′-chimeric RNA or cDNA may be labeled at or near the 5′-end, and is then referred to as a “5′-labeled chimeric RNA” or cDNA. The junction between the 5′-portion and the acceptor portion is termed the “chimeric junction”. A “5′-3′-chimeric RNA” is a 5′-chimeric RNA having an additional 3′ portion (that may be RNA, DNA, etc.) that is trans-spliced, ligated or otherwise affixed at or near the 3′ end of the acceptor RNA transcript. The acceptor RNA transcript may be processed to remove, for example, poly-A sequence prior to addition of the 3′ portion.

The term “complementary DNA” or “cDNA” includes any sense or antisense DNA copy of an RNA, particularly an RNA produced by in vitro or in vivo transcription. A cDNA may contain a copy of only a portion of an RNA. For example, a fragment of cDNA released by digestion with a tagging enzyme (a TAG, as described herein) is a cDNA. A cDNA may be single or double stranded. A portion of a cDNA is said to “correspond to” or “be derived from” an RNA when it comprises sense or antisense sequence of that RNA.

The term “including” is used herein to mean, and is used interchangeably with, the phrase “including but not limited to”.

The term “intron” is used herein to refer to any sequence that is transcribed into an RNA and later removed by a splicing reaction. Introns may occur between exons in a transcript or before the first exon or after the last exon. The portion of a 5′-trans-splicing RNA that is removed during the trans-splicing process is referred to as an intron.

The term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. An “oligonucleotide” is a polymer comprising between 5 and 500 nucleotides and/or analogs thereof.

The term “percent identical” refers to sequence identity between two amino acid sequences or between two nucleotide sequences. Percent identity can be determined by comparing a position in each sequence which may be aligned for purposes of comparison. Expression as a percentage of identity refers to a function of the number of identical amino acids or nucleic acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

A “transcript” is any RNA produced, at least in part, by the action of an RNA polymerase. Examples of transcripts include mRNAs, rRNAs, tRNAs and other small RNAs, such as those involved in splicing.

2. 5′-Trans-Splicing Nucleic Acids and Products

In certain aspects, the disclosure provides 5′-trans-splicing nucleic acids for affixing an exon sequence to the 5′ ends of acceptor RNA species in vivo or in an in vitro trans-splicing reaction. A 5′-trans-splicing nucleic acid comprises an exon and an intron, arranged so that the exon is upstream (5′ relative to) the intron. When a 5′-trans-splicing nucleic acid is expressed as an RNA in a cell, the exon sequence is transferred, via a trans-splicing reaction, to the 5′ end (or near the 5′-end) of another RNA (“acceptor RNA”) produced by the cell. The term “5′-trans-splicing nucleic acid” is intended to include an RNA comprising the intron and the exon that undergoes a trans-splicing reaction, as well as any DNA that encodes such an RNA, and any RNA or DNA antisense thereof. The phrase “a cell expressing a 5′-trans-splicing nucleic acid” is intended to include cells that are presently transcribing such a nucleic acid as well as cells that have in the past (or are descended from or fused to such cells) expressed such a nucleic acid and still contain transcripts thereof.

In certain embodiments, an intron of a trans-splicing nucleic acid comprises sequence that, when present in a single-stranded RNA transcript, folds to form secondary structure comprising three hairpin stem-loops. Optionally, the most 5′ of these loops contains the splice donor site in an A form helix. In certain embodiments, the intron comprises a conserved G-T (or G-U in an RNA) immediately downstream of the last nucleotide of the exon. The G forms a 5′-2′ link with the A of the branchpoint in the intron sequence of the acceptor RNA to which the exon will be affixed. Trans-splicing has been observed in a variety of organisms, including cnidarians (e.g. Hydra), nematodes (e.g. C. elegans) and chordates (e.g. ascidians), and an intron from a naturally occurring 5′-trans-splicing nucleic acid of one of these organisms may be employed in methods described herein. For example, SL-1 is a 5′-trans-splicing nucleic acid of C. elegans, and the SL-1 exon is spliced onto approximately 70% of the mRNAs produced by that organism. The SL-1 intron sequence (SEQ ID NO:2, shown in Table 1 below) may be used as the intron portion of a 5′-trans-splicing nucleic acid. SL-2 is another 5′-trans-splicing nucleic acid from C. elegans. The SL-2 intron sequence (SEQ ID NO:5, shown in Table I below) may also be used as the intron portion of a 5′-trans-splicing nucleic acid. Optionally, a sequence that is at least 80% identical to SEQ ID NO:2 or 5 may be used as the intron portion of a 5′-trans-splicing nucleic acid, and preferably the sequence is at least 85%, 90%, 95% or 99% identical to SEQ ID NO:2 or 5.

In general, the exon has little effect on the splicing efficiency of a 5′-trans-splicing nucleic acid. Accordingly, essentially any sequence may be selected as an exon for a 5′-trans-splicing nucleic acid, except that when combined with the intron, the 5′-trans-splicing nucleic acid should be competent to undergo a trans-splicing reaction with an acceptor RNA to yield a trans-spliced product. In certain embodiments, an exon is designed so as to be devoid of splicing consensus sequences (GT-Xn-Yn-Xn-AG, where Xn is an indeterminate number of nucleotides and Yn is a branchpoint nucleotide, often an A) to prevent cryptic aberrant trans-splicing reactions.

In certain embodiments, it is preferable that the exon have at least a portion of known sequence that can be used in designing an oligonucleotide that hybridizes to the exon or to a complement of the exon. The oligonucleotide may be used to prime a polymerase-driven replication of a 5′-chimeric RNA containing the trans-spliced exon at the 5′ end, and the oligonucleotide may contain one or more labels for introducing one or more labels into the replicated nucleic acids. In certain embodiments, the exon itself will contain a label that is a label sequence that is then transferred along with the exon to an acceptor RNA to provide a trans-splicing product containing the label sequence near the 5′ end.

An exon portion of a 5′-trans-splicing nucleic acid may comprise a label. A label, as the term is used herein with respect to nucleic acids, may be a label sequence, an attached label and/or an incorporated label. An exon portion of a 5′-trans-splicing nucleic acid will, if it comprises a label, preferably comprise a label sequence. A label sequence is generally a sequence that makes the exon particularly detectable or useful for some downstream purpose. A label sequence may be a sequence motif that is recognized by a protein or other reagent that binds, modifies or cleaves a nucleic acid. In certain embodiments, a label sequence is a sequence that is recognized by a cleavage reagent, such as a restriction enzyme. Optionally, the cleavage reagent that recognizes the label sequence is one that has a cleavage site positioned external to the recognition site, and preferably in the 3′ direction. In certain preferred embodiments, the recognition site is positioned at or near the 3′ end of the exon, and the cleavage reagent cleaves within the downstream sequence to which the exon has been spliced. In certain embodiments, the cleavage reagent cleaves at a position at least 7, 10 or 14 base pairs distant in the 3′ direction from the 3′-end of the recognition site. In certain embodiments, the cleavage reagent is a restriction enzyme, and optionally a type IIs or type III restriction enzyme, examples of which are presented in Table 3. In certain embodiments, the cleavage reagent is a triple-helical cleavage reagent, connected to, for example, a topoisomerase, such as a topoisomerase I. In certain embodiments, a cleavage reagent is a protein, such as Ku DNA end binding protein (Paillard et al., Nucleic Acids Research (1991), 19, 5619-5624; de Vries et al., Mol. Biol. (1989), 208, 65-78; Gottlieb et al., Cell (1993), 72, 131-142; Walker et al., Nature (2001), 412, 607-14), that binds to a DNA end and protects a defined area of nucleic acid from cleavage with a reagent such as methidiumpropyl-EDTA and Fe-EDTA (MPE-Fe) or DNAse I. In certain embodiments, the label sequence is a sequence motif for binding to a transcription factor or DNA or RNA polymerase, or a motif for a DNA methylase. In certain embodiments, the label sequence encodes a polypeptide label that is translated as an N-terminal fusion protein with the downstream coding sequence to which the exon has been spliced. A polypeptide label may be, for example, a detection label that facilitates detection of a fusion protein, or a purification label, that facilitates purification of a fusion protein. Examples of detection labels include fluorescent proteins (e.g. Green Fluorescent Protein and the many variants thereof), enzymes that catalyze the production of fluorogenic or chromogenic products (e.g. beta-galactosidase, beta-glucuronidase), enzymes that catalyze the destruction of fluorogenic or chromogenic products and epitope tags (short amino acid sequences that are specifically recognized by established monoclonal antibodies, including a myc tag, FLAG tag or VSV tag). Examples of purification labels include polyhistidines (especially hexahistidine sequences, glutathione-S-transferase, thioredoxin, chitin-binding protein, cellulose binding protein and epitope tags (as described above). A polypeptide label may be both a detection label and a purification label.

In certain embodiments, the SL-1 exon sequence (SEQ ID NO:1, shown in Table 1 below) may be used as the exon portion of a 5′-trans-splicing nucleic acid. The SL-2 exon sequence (SEQ ID NO:4, shown in Table 1 below) may also be used as the exon portion of a 5′-trans-splicing nucleic acid. Optionally, a sequence that is at least 80% identical to SEQ ID NO:1 or 4 may be used as the intron portion of a 5′-trans-splicing nucleic acid, and preferably the sequence is at least 85%, 90%, 95% or 99% identical to SEQ ID NO:1 or 4. SEQ ID NOs:7 and 8 are examples of modified SL-1 (mSL-1) exon sequences that, when combined with native SL-1 intron sequence, are efficiently attached to mRNAs through a trans-splicing reaction. Optionally, a sequence that is at least 80%, 85%, 90%, 95% or 99% identical to a sequence selected from the group consisting of SEQ ID NOs: 7 and 8 may be used as an exon for a 5′-trans-splicing nucleic acid. In view of this disclosure, one of skill in the are will appreciate that other modified SL-1 and SL-2 exons may be designed.

In certain embodiments, a 5′-trans-splicing nucleic acid is the complete SL-1 sequence of SEQ ID NO:3. In certain embodiments, a 5′-trans-splicing nucleic acid is the complete SL-2 sequence of SEQ ID NO:6.

TABLE 1
Examples of 5′-trans-splicing sequences

| Sequence Name | Nucleotide Sequence | |
|---|---|---|
| C. elegans SL-1 Exon | GGTTTAATTA CCCAAGTTTG AG | (SEQ ID NO:1) |
| C. elegans SL-1 Intron | GTAAACATTG AAACTGACCC AAAGAAATTT | (SEQ ID NO:2) |
| | GGCGTTAGCT ATAAATTTTG GAACGTCTCC | |
| | TCTCGGGGAG ACAAAAATAC TAA | |
| C. elegans SL-1 Exon/Intron | GGTTTAATTA CCCAAGTTTG AGGTAAACAT | (SEQ ID NO:3) |
| | TGAAACTGAC CCAAAGAAAT TTGGCGTTAG | |
| | CTATAAATTT TGGAACGTCT CCTCTCGGGG | |
| | AGACAAAAAT ACTAA | |
| C. elegans SL-2 Exon | GGTTTTAACC CAGTTACTCA AG | (SEQ ID NO:4) |
| C. elegans SL-2 Intron | GTACGCTGGA GTTCTGACCT TTCGAAAGAA | (SEQ ID NO:5) |
| | AGTGTCAAAC GACTTTAATT TTTGGAACCG | |
| | CTCTGCTGGG GTCATCCGGT AGAGCAAA | |
| C. elegans SL-2 Exon/Intron | GGTTTTAACC CAGTTACTCA AGGTACGCTG | (SEQ ID NO:6) |
| | GAGTTCTGAC CTTTCGAAAG AAAGTGTCAA | |
| | ACGACTTTAA TTTTTGGAAC CGCTCTGCTG | |
| | GGGTCATCCG GTAGAGCAAA | |
| mSL-1a Exon | CCTTACCTGT CTGCCTAACT CCTTCCGAGG | (SEQ ID NO:7) |
| | ATCCACGGAT GGCGGAAGAG TGCAG | |
| mSL-1b Exon | CCTTACCTGT CTGCCTAACT CCTTCCGAGG | (SEQ ID NO:8) |
| | ATCCACGACG GGCGGAAGTT TGAG | |

The ability of a 5′-trans-splicing nucleic acid to participate in a trans-splicing reaction may be assessed in a variety of ways, many of which are known in the art. For example, the 5′-trans-splicing nucleic acid in question may be expressed in a cell culture or in an organism, and the RNAs isolated and probed for the presence of the exon portion of the 5′-trans-splicing nucleic acid. Example 2, below, provides a protocol for assessing the ability of a 5′-trans-splicing nucleic acid to participate in trans-splicing in C. elegans.

In certain embodiments, the disclosure provides nucleic acid constructs comprising a 5′-trans-splicing nucleic acid and a promoter, wherein the promoter is positioned such that it stimulates expression of the 5′-trans-splicing nucleic acid under appropriate conditions. The term “operably linked” is used herein to describe this relationship between a promoter and a 5′-trans-splicing nucleic acid. In certain embodiments, the promoter is a promoter designed to work in a particular organism or class of organisms, such as chordates, vertebrates, mammals, primates, humans, rodents, mice, invertebrates, arthropods, insects, members of the genus Drosophila, D. melanogaster, nematodes, members of the genus Caenorhabditis, C. elegans, C. briggsae, trematodes, cnidarians, ascidians, Protozoa, trypanosomes, plants and fungi. A promoter may be a constitutive promoter, meaning that the promoter is expressed at a relatively constant level in most of the cell types of a particular organism or class of organisms. A promoter may be specific (or relatively specific) to a particular lineage or type of cell. Any promoter with a characteristic expression pattern that differs between cell types will be referred to herein as a “cell-specific promoter”, even though the promoter may be expressed in more than one cell type. Often, a cell-specific promoter may be generated by combining an enhancer element associated with the desired expression pattern with core promoter sequences that require an enhancer element to achieve significant transcription. For example, promoters that are specific to a number of different tissues in C. elegans have been characterized. Transcription may be targeted to C. elegans neuronal cells by using a promoter that comprise an aex-3 enhancer element (Iwasaki et al. 1997). The unc-54 enhancer element targets muscle cell expression (Waterston et al. 1982). Vulval cells/male-specific tail cell expression may be achieved with the lin-31 enhancer element (Tan et al. 1998). Promoters with similar cell-specific expression patterns are well-known in mice, humans, D. melanogaster and other organisms. A promoter may also be a conditional promoter. A conditional promoter is a promoter that is regulated by a molecule or other condition that is controlled by an experimenter. Commonly, a conditional promoter is induced or repressed in response to a molecule. For example, a variety of tet promoters have been designed for use in mice and other organisms that are either repressed or activated when tetracycline is administered to the organisms or the cells. Conditional promoters may also be temperature sensitive. Because many promoters are modular in nature, elements of conditional and cell-specific promoters may be combined to create constructs that express a 5′-trans-splicing nucleic acid in conditions that are controlled both by the cellular environment and the experimenter.

In certain embodiments, the disclosure provides vectors comprising a 5′-trans-splicing nucleic acid, operably linked to a promoter. The term “vector” refers to a nucleic acid molecule that can be introduced into a cell. One type of vector is an episome, i.e., a nucleic acid capable of extra-chromosomal replication. Another type of vector is an integrative vector that is designed to recombine with the genetic material of a host cell. An integrative vector may be designed so that the entire vector integrates or so that only a portion of the vector integrates. An integrative vector may comprise elements of a transposon. Vectors may be both autonomously replicating and integrative, and the properties of a vector may differ depending on the cellular context. For example, a vector may be autonomously replicating in one host cell type and purely integrative in another host cell type. A vector designed to replicate autonomously in a particular organism will generally comprise an origin of replication that functions in that species. A vector may also be designed for transient transfection, generally meaning that the vector neither integrates nor replicates efficiently, and the vector is lost from cells some period of time after transfection. Regardless of the experimental organism the vector is intended for use in, many vectors are assembled using molecular biology techniques and bacteria (particularly Escherichia coli). Therefore many vectors contain an origin of replication that functions in bacteria, such as the pBR322 ori (low copy number) and the pUC ori (high copy number). Many vectors for use in mammals are viral vectors, meaning that the vector contains sequences that allow the vector to be package and delivered to a cell by a viral particle. The viral vector need not, and generally should not, contain sequences to carry out a full viral life-cycle. Examples of viral vectors include adenovirus vectors, adenovirus-associated virus vectors (AAV), lentiviral vectors and herpes virus vectors. Certain viral vectors deliver nucleic acid to the cell as a DNA, while others (e.g. retroviruses) deliver the nucleic acid to the cell as an RNA. Accordingly, the disclosure contemplates DNA and RNA vectors, both in double or single-stranded forms. Additional sequences that may also be included in a vector include transcription termination signals, polyadenylation signals and selectable markers (optionally a vector may include a different selectable marker for each organism in which it is to be introduced). A vector may also be designed to allow controlled integration and or excision of the nucleic acid into the host cell genome. Such control may be provided by placing one or more recombinase sites (e.g. lox sites) at one or more flanking position relative to the 5′-trans-splicing nucleic acid, and providing a nucleic acid encoding a recombinase gene (e.g. Cre recombinase) that is expressed in the desired conditions. In general, vectors can be constructed using techniques well known in the art (Sambrook et al., 1989, Molecular Cloning, A Laboratory Manual, Cold Spring Harbor Press, Plainview N.Y.; Ausubel et al., 1989, Current Protocols in Molecular Biology, John Wiley & Sons, New York N.Y.).

In certain embodiments, the disclosure provides cells comprising a 5′-trans-splicing nucleic acid. A 5′-trans-splicing nucleic acid may be present in a cell as a transcript and/or as a nucleic acid construct operably linked to a promoter. A nucleic acid construct comprising a 5′-trans-splicing nucleic acid may be integrated into a host chromosome or it may be present as a separate episome. In certain embodiments, a cell comprising a 5′-trans-splicing nucleic acid is a cell that is the subject of inquiry, such as a cell of a chordate, a vertebrate, a mammal, a primate, a human, a rodent, a mouse, an invertebrate, an arthropod, an insect, a member of the genus Drosophila, a D. melanogaster, a nematode, a member of the genus Caenorhabditis, a C. elegans, a C. briggsae, a trematode, a cnidarian, an ascidian, a Protozoan, a trypanosome, a plant and a fungus. A cell may be in essentially any context, such as, present in a living or dead organism, or in a tissue sample, or the cell may be present in a cell culture. A cell may also be modified in some way, such that although it is derived from an organism, it is not a type of cell naturally found in any organism. For example, many immortalized cell lines are genetically modified. In certain embodiments, a cell comprising a 5′-trans-splicing nucleic acid is a cell used to propagate or store the nucleic acid, often as a vector. Such cells may be bacteria, such as E. coli or Bacillus subtilis, fungal, such as Saccharomyces cerevisiae, or any other cell type that permits stable maintenance of the nucleic acid.

In certain embodiments, the disclosure provides transgenic organisms that comprise a 5′-trans-splicing nucleic acid. Examples of such organisms include any of those listed above with respect to cell types. In certain embodiments, a transgenic organism provided herein will have a predictable phenotype, which is that a measurable amount of the mRNAs in at least one cell type of the animal will be found to have an exon from the 5′-trans-splicing nucleic acid attached at the 5′ end. Particularly preferred transgenic organisms include mice and nematodes. No claim herein should be interpreted as encompassing a human being, and where an animal is claimed as such, it should be understood to be a non-human animal.

3. Cell-Based Methods for Making 5′-Chimeric RNAs and cDNAs

In certain aspects, the disclosure provides cell-based methods for making an RNA or complementary DNA (cDNA) that has an additional sequence at the 5′ end, termed “5′-chimeric RNA” or “5′-chimeric cDNA”. In certain embodiments, methods described herein comprise causing a cell or population of cells to express a 5′-trans-splicing nucleic acid. The 5′-trans-splicing RNA that is produced participates in a trans-splicing reaction with another RNA in the cell (called the acceptor RNA), such as an mRNA, and that the exon of the 5′-trans-splicing RNA is transferred to the acceptor RNA and becomes the 5′-end sequence thereof. In certain embodiments, the sequence of the exon is known, and optionally engineered to contain one or more useful label sequences, and accordingly, the 5′-end of these RNAs is amenable to further analysis. The cell or population of cells in which the 5′-trans-splicing nucleic acid is expressed may be cultured cell(s), cell(s) in a tissue sample or cell(s) in an organism. The 5′-trans-splicing nucleic acid may be expressed in a cell-specific manner or it may be generally expressed in all or most of the cells.

In certain embodiments, RNA is prepared from cells expressing a 5′-trans-splicing nucleic acid. A variety of methods for obtaining an RNA preparation from cells are known in the art. For example, total RNA may be prepared according to a standard RNAse-free phenol-chloroform extraction protocol. Alternatively, a number of kits are available for isolation of total RNA. An RNA preparation enriched for poly-A RNA may be prepared by contacting a total RNA preparation or a cellular extract with oligo-dT (poly-thymidine) oligonucleotides that are, for example, adhered to a solid support (e.g. a bead or resin), so that poly-A RNAs also become adhered to the solid support. An affinity column of oligotex beads (dC10T30 oligonucleotides covalently linked to the surface of polystyrene-latex particles) may be used to purify mRNA from total RNA. Oligonucleotides containing an amino group (NH2) at their 3′-ends may be covalently coupled to N-hydroxysuccinimide (NHS)-agarose (Hammarsten and Chu 1998).

In certain embodiments, an RNA preparation is enriched for RNAs containing the exon portion of a 5′-trans-splicing nucleic acid by contacting a mixture of RNAs with an “exon purification oligonucleotide”. An exon purification oligonucleotide is an oligonucleotide that hybridizes to an exon, and typically comprises a portion of sequence that is complementary to at least a portion of the exon sequence. The complementary oligonucleotide may be adhered to a solid, semi-solid or insoluble support, e.g. to form an oligonucleotide-based capture medium, so that RNAs containing an exon portion of the 5′-trans-splicing nucleic acid are selectively bound. This enrichment process provides for enrichment of appropriately trans-spliced RNAs from a complex mixture of RNAs, and it is particularly useful in applications wherein the 5′-trans-splicing nucleic acid is expressed in only a fraction of cells. This enrichment step combined with the use of cell-specific promoters for the expression of 5′-trans-splicing nucleic acids permits the selective isolation of RNAs expressed in selected cell types even in complex biological materials, such as tissue samples and whole organisms without the use of time-consuming cell-sorting techniques.

In certain embodiments, an RNA preparation may be depleted of RNAs containing the intron portion of a trans-splicing nucleic acid (e.g. 5′-trans-splicing RNAs that have not participated in a trans-splicing reaction). If RNAs containing an intron portion of a trans-splicing nucleic acid are not removed, they may represent a substantial portion of the RNA used in later analytical steps, making these later analytical steps more difficult. The depletion may be accomplished by contacting a mixture of RNAs with an “intron purification oligonucleotide”. An intron purification oligonucleotide is an oligonucleotide that hybridizes to an intron, and typically comprises a portion of sequence that is complementary to at least a portion of the intron sequence. The complementary oligonucleotide may be adhered to a solid support, e.g. to form an oligonucleotide-based capture medium, so that RNAs containing an intron portion of the 5′-trans-splicing nucleic acid are selectively bound and discarded. In certain embodiments, both an exon-enriching procedure and an intron-depletion procedure are employed. In either the intron or exon affinity purification procedures, the oligonucleotide may be bound to a biotin moiety and captured on streptavidin beads (e.g. streptavidin magnetic beads) along with the nucleic acid hybridized to the oligonucleotides. Hybrids of RNA and biotinylated oligonucleotides may also be captured onto a PCR tube coated with streptavidin. These oligonucleotide-based enrichment and depletion procedures may also be used after RNA has been converted to cDNA.

In certain embodiments, RNAs are used as templates to produce cDNAs. The term “cDNA”, as used herein, includes any sense or antisense copy of an RNA, particularly an RNA produced by in vitro or in vivo transcription. A cDNA may contain a copy of only a portion of an RNA. Often, a cDNA is a double-stranded cDNA, meaning that it contains a sense strand and an antisense strand. When the terms “5′” and “3′” are used in reference to a double-stranded cDNA, they are used in reference to the sense strand. Accordingly, the 5′-end of a double-stranded cDNA corresponds to the 5′ end of the RNA from which it is derived. Any cDNA that traces back through one or more steps of copying to an RNA is considered to have been “derived from” that RNA, even where the copying process introduces sequence changes. Likewise, if an RNA is obtained from a cell, any nucleic acid derived from that RNA is considered to have been derived from the cell. In general, cDNA synthesis involves making a first, antisense strand using the RNA itself as a template. Reverse transcriptase uses an oligonucleotide primer that hybridizes to the RNA in order to initiate the synthesis of the first cDNA strand. The primer for first-strand synthesis may be termed a “downstream” primer, as it is typically designed to hybridize in the 3′-region of the RNA. A downstream primer is often a poly-T primer that initiates reverse transcription from the poly-A tail found at the 3′-end of many RNAs, particularly mRNAs. Reverse transcription can also be primed with a cocktail of random hexamers (or higher order multimers, e.g. septamers, octamers) that prime reverse transcription from random and different positions within each RNA. Second-strand synthesis can be primed using the natural tendency of reverse transcriptase to form a hairpin loop when it reaches the 5′ end of the RNA. Alternatively, since the desired 5′-chimeric RNAs will have a trans-spliced exon of known sequence at the 5′-end, the first strand antisense cDNA will contain an antisense exon sequence at the 3′-end. An oligonucleotide primer that hybridizes to this sequence (e.g. an oligonucleotide that has a sequence identical or nearly identical to at least a portion of the trans-spliced exon) may be used to prime synthesis of the second strand of cDNA. Additional cDNA copies may be generated by single strand amplification using either a poly-T primer (to copy the antisense strand) or an oligonucleotide primer that hybridizes to the trans-spliced exon (to copy the sense strand). Both primers may be used for exponential amplification, e.g. by polymerase chain reaction (PCR). Note that because the known exon sequence is positioned at the 5′-end, cDNA synthesis using the exon sequence to prime synthesis will generate cDNAs with complete 5′ sequences.

The oligonucleotides used in making first and second strand cDNAs, as well as later cDNA copies, may be used to introduce labels and to replicate label sequences already present in the 5′-chimeric RNAs or cDNAs. In certain embodiments, the label is a sequence motif, such as a recognition site for a protein or other reagent that binds to, modifies or cleaves DNA. In preferred embodiments, the label is a recognition sequence for a cleavage reagent, and optionally a cleavage reagent that cleaves at a distance from the recognition site (i.e. cleaves outside of the recognition sequence). In certain embodiments, the cleavage reagent cleaves at a distance of at least 7, 10 or 14 base pairs from the recognition site (i.e. at least a certain distance upstream of the 5′-end of the recognition site and/or at least a certain distance downstream of the 3′-end of the recognition site). In certain preferred embodiments, the cleavage reagent is a restriction enzyme. Examples of preferred restriction enzymes include type IIs restriction enzymes (e.g. Bpm I, Bsg I and Mme I) and type III restriction enzymes (e.g. EcoP15I). In certain embodiments, the cleavage reagent is a triple-helical cleavage reagent, connected to, for example, a topoisomerase, such as a topoisomerase I. In certain embodiments, a cleavage reagent is a protein, such as Ku DNA end binding protein (Paillard et al., Nucleic Acids Research (1991), 19, 5619-5624; de Vries et al., Mol. Biol. (1989), 208, 65-78; Gottlieb et al., Cell (1993), 72, 131-142; Walker et al., Nature (2001), 412, 607-14), that binds to a DNA end and protects a defined area of nucleic acid from cleavage with a reagent such as methidiumpropyl-EDTA and Fe-EDTA (MPE-Fe) or DNAse I (in other words, the protecting protein and the reagent that cleaves nucleic acid should be viewed together as a “cleavage reagent”, as the term is used herein). Similarly, a cleavage reagent may be a relatively non-specific nuclease and a nucleic acid that binds to and protects the 5′ portion of the 5′-chimeric nucleic acid from digestion by the nuclease. In certain embodiments, an oligonucleotide primer may have an incorporated or attached label. An incorporated label is a label that is part of the nucleic acid polymer, such as a base analog or an isotopic atom. An attached label is a moiety that is covalently or non-covalently associated with the nucleic acid polymer, most often through a bond with a nitrogen or oxygen of the nucleic acid polymer. In some instances, it may be difficult to discern whether a label is incorporated or attached, and therefore, the phrase “incorporated or attached label” is intended to include all labels that are stably associated with or part of the labeled nucleic acid. In certain embodiments, an incorporated or attached label is an affinity purification label, such that nucleic acids associated with the affinity purification label are readily separated from nucleic acids that do not contain the affinity purification label. Nucleic acids associated with an affinity purification label may be separated by, for example, contacting the nucleic acids with a capture medium comprising an agent that binds specifically to the affinity purification label. Biotin is an example of an affinity purification label that is commonly used, and biotin may be bound to a capture medium comprising streptavidin. Other affinity purification labels include digoxigenin, a peptide attachment, a peptide nucleic acid (PNA) attachment and a cholesterol attachment. An incorporated or attached label may also be a detection label, such that nucleic acids associated with the detection label are detectably distinct from nucleic acids that do not contain detection label. Examples of commonly used detection labels include radioactive isotopes, such as ³²P and ³⁵S, and fluorophores, such as Cy-3 and Cy-5. In certain preferred embodiments, an oligonucleotide is used to produce cDNAs that have both a cleavage reagent recognition site and an affinity purification label. In certain embodiments, the sequence of the exon of the 5′-trans-splicing nucleic acid is changeable, and therefore the exon may be designed at the outset with desired sequence elements, such as a cleavage reagent recognition site or a protein binding site. Labels may be similarly added to the 3′ end of a cDNA by using a labeled poly-T primer. In certain embodiments, the oligonucleotide primer is designed to introduce a sequence change into the cDNA. For example, an oligonucleotide may introduce a label sequence that was not originally present in the 5′-chimeric cDNA or 5′-chimeric RNA.

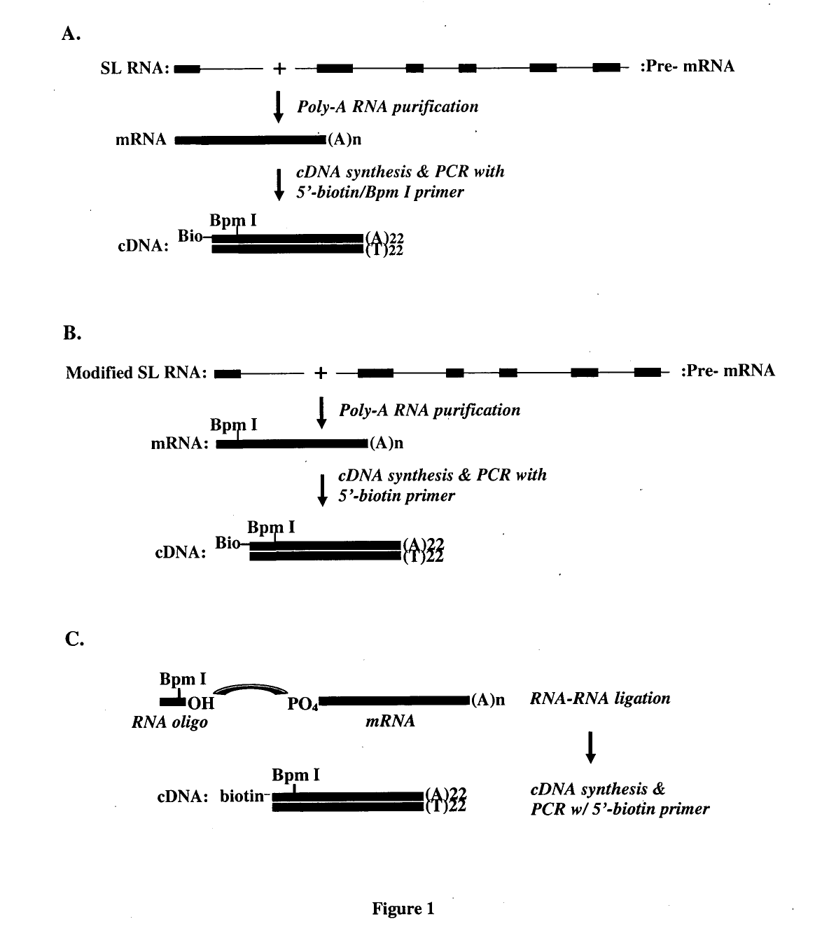

In certain embodiments, a label sequence or other desirable sequence motif is already present in the 5′-chimeric cDNA or 5′-chimeric RNA (e.g. the sequence motif may be introduced in vivo as part of the trans-spliced exon sequence), and an oligonucleotide simply serves to copy the sequence motif. Examples of methods for producing 5′-labeled chimeric cDNAs are shown in FIG. 1.

In many embodiments, the methods described herein for producing 5′-chimeric RNAs and cDNAs, and 5′-labeled chimeric RNAs and cDNAs are used to analyze populations of RNAs (meaning more than one RNA species, and typically hundreds or thousands of RNA species). Accordingly, populations of 5′-chimeric RNAs and cDNAs may be generated that vary through the 3′ portion of the sequence derived from acceptor RNAs made by a cell, but having a uniform (or nearly uniform) 5′-end, corresponding the trans-spliced exon, or a portion thereof. However, the methods described herein may also be used to analyze single RNA species. For example, the methods described herein may be used to identify the 5′-end(s) of an RNA previously identified only by an internal EST sequence.

The term “label” as used herein is intended to mean any chemical entity or sequence motif that can be used to detect, purify, modify, cleave or bind a protein to a labeled nucleic acid. The term “label” is used to include functional sequence elements, such as a sequence elements that are recognized by a protein that cleaves, modifies or binds nucleic acids. The term “label” is also used to include non-nucleic acid moieties, such as biotin, radioactive isotopes and fluorophores that are covalently or non-covalently attached to a nucleic acid of interest.

5′-trans-splicing nucleic acids may also be used in a cell-free system, wherein RNAs (e.g. poly-A RNAs from a cell of interest) are incubated with a 5′-trans-splicing RNA and the various splicing factors involved in performing a trans-splicing reaction. These trans-splicing factors may be provided as purified proteins or as a nuclear extract, such as a nuclear extract from C. elegans.

4. In Vitro Methods for Making 5′-Chimeric RNAs and cDNAs

In certain aspects, the disclosure provides in vitro methods for making an RNA or complementary DNA (cDNA) that has a chimeric sequence at the 5′ end. In certain embodiments, methods described herein comprise attaching an oligonucleotide to the 5′ end of an acceptor RNA in vitro, thereby creating a 5′-chimeric RNA. In certain embodiments, the sequence of the oligonucleotide is known, and optionally engineered to contain one or more useful sequence motifs, and therefore the 5′-portions of these RNAs are amenable to further analysis. The RNA that is treated in this manner may be a single species of RNA or an RNA preparation comprising more than one species of RNA. In preferred embodiments, the RNA preparation is whole or poly-A RNA from a cell culture, tissue or organism of interest. Optionally the oligonucleotide is an single-stranded RNA, a single-stranded DNA, a single-stranded RNA-DNA hybrid, or a duplex or triplex combining any of the preceding.

In certain embodiments, ligation of an oligonucleotide to the 5′ end of an RNA comprises selective ligation of the oligonucleotide to RNA molecules that have an intact 5′-cap structure. Most mRNAs in eukaryotes have at their 5′-ends a structure referred to as a “cap” that generally consists of an m₇G (guanosine methylated at the 7 position) connected to the 5′-end of the RNA by a chain of phosphates. An oligonucleotide can be ligated to an RNA having a free phosphate at the 5′-end, using a ligase such as T4 RNA ligase. RNA ligases will not ligate the oligonucleotide to capped RNAs. Accordingly, in certain embodiments, the cap may be digested using a phosphatase such as tobacco acid pyrophosphatase (TAP), to reveal a free phosphate at the 5′-end of the RNA. In a mixture of RNAs obtained from a cell, there will generally be capped RNAs and RNAs that do not have caps. The RNAs without caps are often degraded or otherwise undesirable RNAs. In certain preferred embodiments, the oligonucleotide is selectively ligated to the 5′-ends of capped RNAs. Often the RNAs without caps will have a 5′ phosphate, meaning that an oligonucleotide could be readily attached by ligation Accordingly, the mixture of RNAs may be treated with a phosphatase, such as calf intestinal phosphatase (CIP) that removes phosphates from the 5′-ends of uncapped RNAs. Then the mixture may be treated with an enzyme that removes the caps to leave a 5′-phosphate, followed by treatment with the RNA ligase. Because the uncapped RNAs were treated with a phosphatase, these RNAs do not react with the ligase.

The oligonucleotide to be added by an in vitro procedure may have essentially any desired sequence. In certain embodiments, the oligonucleotide comprises a sequence element or other label as described above with respect to the cell-based system for generating 5′-chimeric RNAs. In a preferred embodiment, the oligonucleotide comprises a recognition sequence for a cleavage reagent that cleaves DNA at a position outside of the recognition sequence, and the recognition sequence is preferably positioned at or near the 3′-end of the oligonucleotide (e.g. within the last 10 bases). In certain preferred embodiments, the oligonucleotide is a single-stranded RNA oligonucleotide.

Once the oligonucleotide has been ligated to the acceptor RNA, the ligated product (5′-chimeric RNA, and, if the oligonucleotide comprises a label, 5′-labeled-chimeric RNA) may be treated in the same manner as the trans-spliced RNAs described above. For example, the 5′-chimeric RNA may be separated from other RNAs by affinity oligonucleotide column chromatography using a purification oligonucleotide that hybridizes to the oligonucleotide. The 5′-chimeric RNA may be used to synthesize first and second strand 5′-chimeric cDNAs as well as further amplified products. The sequence of the 5′-chimeric RNA may be altered by the oligonucleotide primer used during cDNA synthesis and/or amplification. The 5′-chimeric RNA may be labeled using a labeled oligonucleotide primer during cDNA synthesis and/or amplification.

5. 5′ RNA End Determination (5′-RED)

In certain embodiments, the disclosure provides methods for analyzing sequence at or near the 5′-ends of RNAs. The disclosure provides a variety of methods for obtaining 5′-chimeric RNAs and 5′-chimeric cDNAs. A 5′-chimeric RNA comprises a portion that corresponds to an acceptor RNA transcript and a 5′-portion having a sequence that is trans-spliced, ligated or otherwise affixed at or near the 5′ end of the acceptor RNA transcript. Any known sequences present at the 5′-portions of these chimeric nucleic acids may be employed in a variety of methods to ascertain the adjacent downstream sequence that corresponds to the acceptor RNA transcript. While not wishing to be bound to theory, it is generally accepted that the exon of a 5′-trans-splicing RNA is spliced onto the acceptor RNA (e.g. an mRNA) at a position having the characteristics of a 3′-splice site (e.g. an A-G sequence), with sequence upstream of the 3′-splice site in the acceptor RNA being eliminated in the splicing reaction. Accordingly, it is expected that the junction between the added exon and the acceptor RNA will not usually correspond to the exact 5′-end of the acceptor RNA. 5′-chimeric RNAs and cDNAs generated in vitro will tend to have the complete 5′-end sequence of the acceptor RNA, and the chimeric junction will tend to correspond to the exact 5′-end.

In certain embodiments, a 5′-chimeric cDNA may be used as template in a Sanger sequencing method, with an oligonucleotide that hybridizes to the known 5′ sequence serving as a sequencing primer. This embodiment permits determination of sequence at or near the 5′-end of the acceptor RNA transcript. However, such a method is labor intensive, and other methods described herein are more compatible with high-throughput analysis.

In certain embodiments, the disclosure provides methods for rapidly ascertaining sequence in a 5′-chimeric cDNA that corresponds to sequence at or near the 5′-end of an acceptor RNA transcript. In certain embodiments a method uses, as starting material, 5′-labeled-chimeric cDNA comprising an affinity purification label such as biotin at or near the 5′-end and comprising a recognition site for a cleavage reagent, optionally a restriction enzyme, that cleaves DNA at a position at least 7, 10 or 14 nucleotides removed in the 3′ direction from the 3′ boundary of the recognition site. This cleavage reagent is referred to as the “tagging” reagent. In certain embodiments, a tagging cleavage reagent is a tagging restriction enzyme. Examples of available tagging restriction enzymes are presented in table 3. Preferably the recognition site is positioned within the 10 bases immediately upstream of the chimeric junction, and in particularly preferred embodiments, the recognition site is positioned immediately upstream of the chimeric junction.

The 5′-labeled-chimeric cDNA is digested with the tagging reagent to produce a small cDNA containing the 5′ affinity purification label followed by the remainder of the chimeric portion and a small portion of unknown sequence corresponding to sequence from the acceptor RNA. The digestion also typically releases at least one unlabeled downstream fragment. The 5′ pieces may be separated from the downstream pieces by application to capture medium, such as beads or chromatographic substrate comprising an agent to binds to the affinity purification label. For example if the affinity purification label is biotin, the 5′ cDNA pieces may be purified by application to a streptavidin matrix, such as magnetic streptavidin beads.

Purified 5′ cDNA fragments may be ligated at the 3′ end with an adapter DNA, preferably containing a cleavage reagent recognition site, optionally a restriction enzyme recognition site, and this cleavage reagent is referred to as the “second anchoring cleavage reagent”. Anchoring cleavage reagents preferably cut within the recognition sequence (unlike the tagging cleavage reagent). Preferred anchoring cleavage reagents are restriction enzymes, most preferably restriction enzymes having a four, five or six base pair recognition sequence. The previous digestion with the tagging cleavage reagent may create a 5′ overhang, a 3′ overhang or a blunt end at the 3′ end of the cDNA fragment, and depending on the type of end created, different strategies may be employed for adding the adapter DNA. A 5′ overhang may be filled by treatment with a polymerase such as T4 DNA polymerase. The adapter DNA may then be added by blunt end ligation. A 3′ overhang may be blunted by an enzyme with specific single-stranded exonuclease activity, such as the exonuclease activity of T4 DNA polymerase. Alternatively, the 3′ overhang may be used to directly introduce the adapter DNA. Because the nucleotides in the 3′ overhang are part of the unknown acceptor RNA sequence, direct ligation may be accomplished by using an adapter DNA that is generated as a pool of DNAs having random nucleotides in the positions that would correspond to the 3′ overhang. In this manner, a portion of the adapters will successfully base pair with the 3′ overhang and be ligated. The ligated cDNA fragments are purified from the free adapter DNA, again using the affinity purification label (or, for example, the known 5′ sequence), and subsequently subjected to PCR amplification with PCR primers. The 3′ primer contains the recognition site for the second anchoring cleavage reagent. The 5′ primer is designed to introduce the recognition site for the first anchoring cleavage reagent. Alternatively, the sequence for the first anchoring cleavage reagent may already be present in the fragment (e.g. it could be present in the original 5′-chimeric RNA or introduced during cDNA synthesis and/or amplification). Preferably, the recognition sites for the first and second anchoring cleavage reagents are immediately upstream and downstream of the small portion of unknown sequence from the acceptor RNA, however, the process may be designed such that some number of base pairs intervene. Such intervening sequence is not favored, however, because the intervening sequence will also be sequenced at the sequencing stage, thereby increasing the cost and effort associated with sequencing.

The amplified products are purified and double-digested with the first and second anchoring cleavage reagents. Then, the double-digested fragments are separated by size. For example, the fragments may be separated on a polyacrylamide gel and the band containing the released fragments (termed TAGs) is eluted from the gel.

Optionally, the purified TAGs are ligated to form concatemers. Preferably concatemers containing 35 to 60 TAGs are then purified and ligated into a plasmid vector. The vectors are transformed into E. coli to be isolated for sequencing. First and second anchoring cleavage reagents may be essentially any cleavage reagents that cleave within their recognition sites. Optionally, the first and second anchoring cleavage reagents are selected to permit directional concatemerization. For example, if one anchoring cleavage reagent makes an overhang, while the other is a blunt cutter, then the concatemer ligation will result in the blunt ends ligating together and the overhang ends ligating together. Both the first and second anchoring cleavage reagents may be selected to leave blunt ends, or both enzymes may be selected to leave the same overhang.

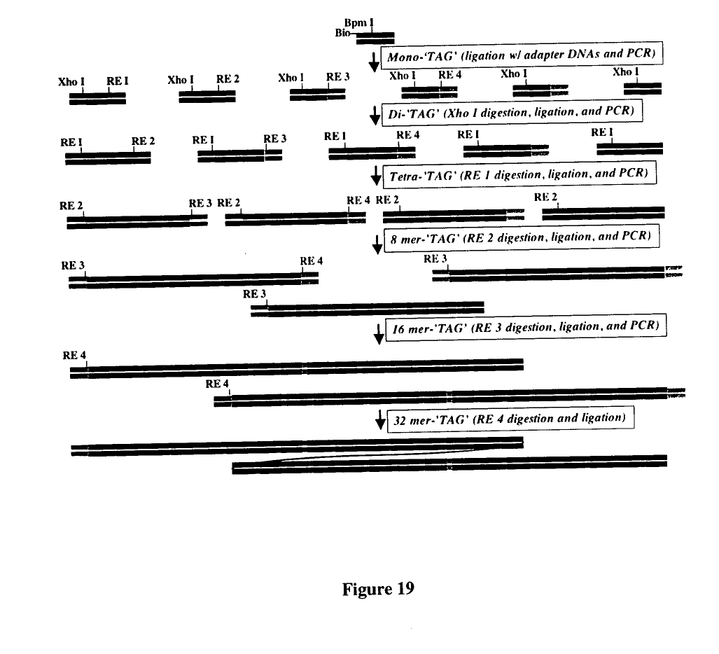

In certain embodiments, concatermerization may involve the use of two or more different 3′ anchoring cleavage reagents. An example of such a procedure employing four different 3′ anchoring cleavage reagents is described in FIG. 19. As a first step, TAGs containing 5′ anchoring cleavage reagent recognition sites are generated. These may then be split into pools, with each pool ligated to a 3′ adaptor having a recognition site for a different 3′ anchoring cleavage reagent. This results in the generation of TAGs that all have the same 5′ anchoring cleavage reagent but different 3′ anchoring cleavage reagents. The same result may be achieved by ligating a single pool of TAGs with a mixture of different 3′ adaptors. TAGs may then be cleaved with the 5′ anchoring cleavage reagent and ligated to form head-to-head ditags having a recognition sequence for a second anchoring cleavage reagent recognition site (generally two different anchoring cleavage agents) at each end. If the TAGs were generated using different pools of TAGs, the exact recognition site at each end can be controlled (e.g., by ligating TAGs having the recognition site for cleavage reagent 1 with those having the recognition site for cleavage reagent 2, etc.). If the TAGs were generated as a single pool, then the combination of recognition sites at either end may be random. In either case, the ditags may be treated with the first of the 3′ anchoring cleavage reagents and ligated again, to form tetra-tags. These are then treated with the second 3′ anchoring cleavage reagents and re-ligated, giving “8mer”-tags, and so on. This directed concatenation approach has proven highly efficient even with somewhat less purified TAG populations, which is advantageous because large, highly pure population of small DNA fragments can be laborious and hinder efforts to handle multiple samples at the same time. Optionally, large-scale PCR reactions and PAGE purification steps may be omitted when using this type of concatemerization protocol.

Concatemers may be sequenced by Sanger-type sequencing methods. Present methods generally use a mix of fluorescently labeled dideoxy nucleotides, and the fragments are resolved on a microcapillary electrophoresis system equipped with a fluorescence reader that generates a chromatogram. Other sequencing techniques may be used. For example, if a probe array containing nucleic acids expected to correspond to different transcripts is available, the TAGs may be contacted with the probe array, and the sequence of each TAG may be deduced by determining the sequence of the probes at the hybridizing positions on the probe array. Typically, the TAGs would be labeled with a detection label, e.g. a fluorescent label, prior to contacting with a probe array. Note that if a probe array or other sequencing by hybridization technique is used, TAGs may be sequenced without using a concatemerization step.

The TAG size is determined by at least three parameters: the distance between the tagging cleavage reagent recognition site and the cleavage site, the proximity of the recognition site to the beginning of the sequence corresponding to the acceptor RNA, and the method by which the 3′ adapter is added. For example, when the 3′ end of the cDNA fragment is prepared for adapter ligation by blunting by exonuclease activity, the TAG size is reduced. Examples of tagging enzymes that cleave to give different TAG sizes are described in Table 3.

TAG size may be selected depending on the size and/or complexity of the genome of the organism to be studied. Generally, the larger and/or more complex a genome is, the larger the TAG size that will be preferable.

TAG sequences may be matched up to genomic sequences; this permits assignment of TAGs to possible transcripts. In certain embodiments, a set of criteria may be used to locate a TAG within the DNA sequencing data and to search for the matching sites (sequences) within the genome sequence of the subject organism. Optionally TAGs may be filtered using various criteria. For TAGs derived from trans-spliced RNAs, a TAG sequence should be located after a splicing acceptor site (AG) within the genome sequence. For TAGs derived from any RNA source, the matched genome sites should be oriented in the same direction as the TAG sequence. If either of these criteria is not met, the TAG is likely to be an artifact and may be discarded. When a TAG sequence derived from a trans-spliced RNA is located more than once within the genome sequence, the matching genome site that follows the conserved splicing acceptor consensus sequence may be selected as the true corresponding genomic site for the TAG. The preceding criteria may be assessed by hand or with the aid of a computer running appropriate software. Computer readable instructions may be placed on a storage medium, including optical or magnetic storage media, such as CD-ROMs, floppy disks and hard drives.

A variety of analytical processes may be used to extract information from the comparison of TAG sequence to genome sequence. In certain embodiments, one or more distance parameters are calculated, and the distance parameters may be used to infer information about the transcript corresponding to the TAG sequence. A trans-splicing reaction often removes a small amount of sequence from the 5′-end of the acceptor RNA, and therefore TAGs from a trans-spliced RNA will tend to correspond to sequence that is slightly internal to the 5′-end After identifying the genome site matched with a TAG sequence, one may search for the gene closest to the site of the TAG sequence in the genome. One distance parameter that may be calculated is the distance from the genome site of the TAG sequence to the first exon (5′-most exon). An additional distance parameter that may be calculated is the distance from the genome site of the TAG sequence to the nearest exon. A further distance parameter that may be calculated is the distance from the genome site of the TAG sequence to the nearest ATG codon of the gene. One or more distance parameters may be used to judge whether the TAG is located at the known 5′-end or not. When the TAG is located at the known 5′-end, the distance to the first exon is generally less than 10. When the TAG comes from an additional transcriptional initiation site in an intron of a known gene, the distance from the genome site to the first exon is large, but the distance to the closest exon should be 1. When a TAG is located far from any known gene, both distance parameters become very large. When the TAG is located at the new 5′-end of a known gene, both distance parameters are also large, but are smaller than those for unknown genes. In certain embodiments, TAGs are classified based on these distance parameters. In certain embodiments, distance parameters may be calculated by hand, or by a computer running appropriate software. TAG sequences may also be individually inspected using short oligo search programs available, for example, from NCBI or WormBase. For the candidates of new genes and new 5′-ends of known genes may also be individually inspected using gene prediction data in each cosmid sequence at NCBI.

In certain embodiments, 5′-RED techniques may be used to count TAGs and generate a relative quantitative measure of the abundance of each transcript in the source cells. In alternative embodiments, 5′-RED may be used with the goal of cataloging different transcripts and particularly identifying rare transcripts. In the latter instance it may be desirable to employ a procedure to equalize (or partially equalize) the abundance of TAGs from abundant transcripts with the abundance of TAGs from rare transcripts. Subtractive hybridization techniques are available for use with RNA populations, and such approaches may be used here, either with RNA pools, cDNA pools or with pools of TAGS.

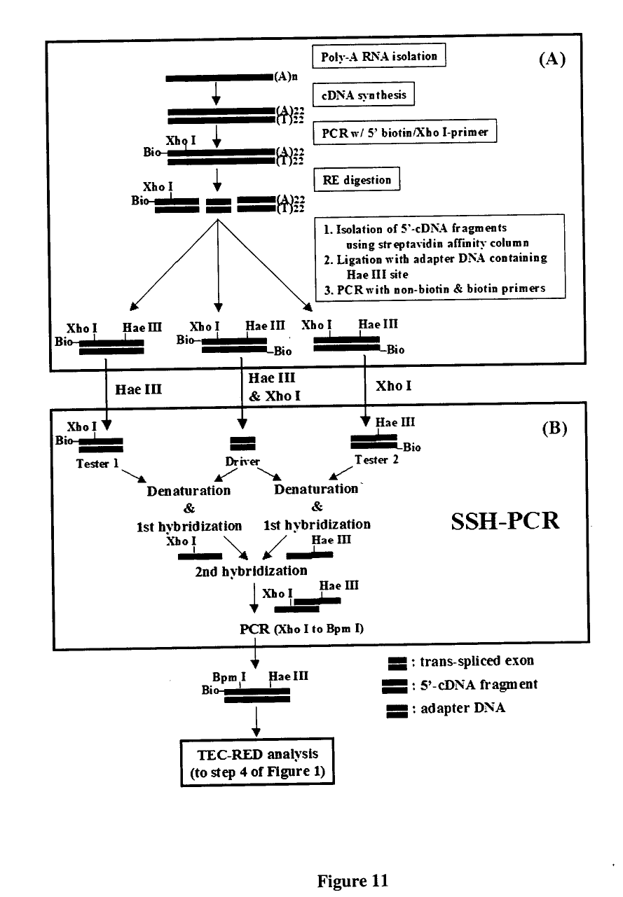

In certain embodiments, the disclosure provides methods normalization of high- and low-abundance RNA or cDNA sequences. The term “normalization” is meant to indicate that a population of RNA or cDNA species having a certain variance in the abundance of each RNA or cDNA species is processed so as to reduce the variance in species abundances. Normalization may be useful in trying to identify rare transcripts because the frequencies at which high and low abundance species occur in a sample are brought closer in value. One approach to accomplishing a normalization involves so-called SSH-PCR. An exemplary embodiment of this technique is illustrated in FIG. 11. In certain embodiments, 5′ cleavage fragments are obtained by digesting 5′-chimeric cDNA with a cleavage reagent, as shown, for example, in box (A) of FIG. 11. In certain embodiments, a 5′ cleavage fragment is used as the “driver”. Generally, a driver is used in an SSH-PCR protocol to hybridize to and remove nucleic acids that hybridize to either strand of the driver. A nucleic acid that is contacted with a driver is a “tester”. Tester nucleic acids that do not hybridize to a driver may be amplified. In certain embodiments, a tester is a “nested 5′ cleavage fragment”, meaning that the 5′ cleavage fragment has an additional sequence at the 5′ or 3′ end that facilitates later amplification of the tester. In certain embodiments, there is a 5′-tester, that has an additional sequence at the 5′-end, and a 3′-tester, that has an additional sequence at the 3′-end. Optionally, both the 5′-testers and the 3′-testers are contacted with the driver (a “free” 5′ cleavage fragment). Those 5′ and 3′-testers that do not hybridize to drivers are mixed together. Hybrids formed by one strand of a 5′-tester and one strand of a 3′-tester are selectively amplified by PCR using primers that hybridize to the additional 5′ and 3′ sequences. One or more steps of an SSH-PCR may be carried out following one or more steps of the protocol provided by CLONTECH (Foster City, Calif.). and the method may be applied to long RNAs or cDNAs, as well as to 5′ cleavage fragments and TAGs themselves. It is expected that an SSH-PCR method applied to long RNAs will perform poorly in normalizing abundant and rare alternative transcripts encoded by a single gene, as these abundant transcript may share substantial sequence identity with the rare transcript. Accordingly, it is expected that by performing SSH-PCR on 5′ cleavage fragments, even alternative transcripts can be successfully normalized. In certain embodiments, the normalized 5′-cDNA fragments may be directly introduced into a 5′-RED analysis.

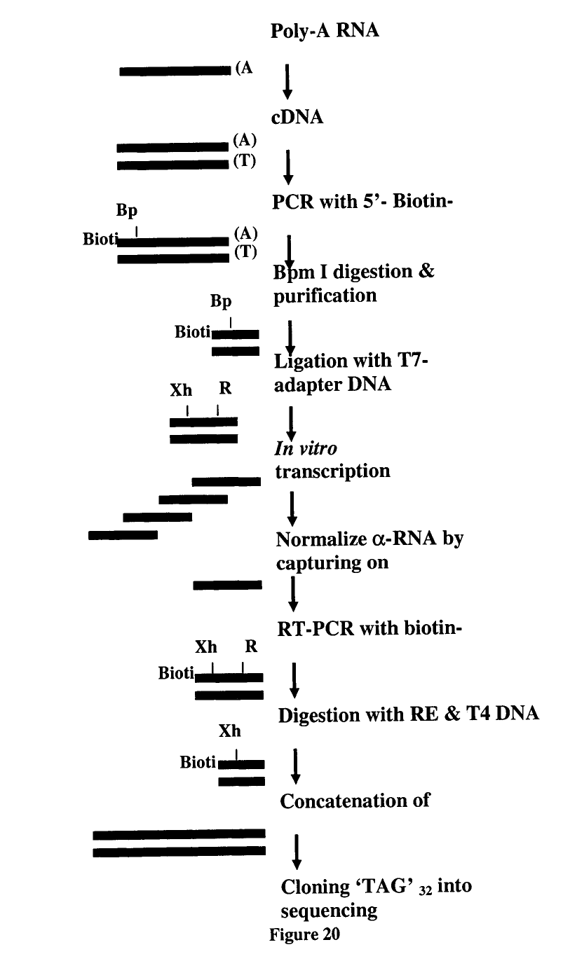

In certain embodiments, normalization may be achieved by using an approach based on hybridization to randomized oligomers. A schematic illustrating an example of such a method for obtaining normalized, concatenated 5′ TAGs is shown in FIG. 20. In this example, the TAGs are ligated with a 3′ adapter containing a T7 polymerase site. This allows the TAGs to be transcribed into single stranded RNAs. These RNAs are then contacted with a set of immobilized (e.g. on beads) random 14mers. The RNAs that bind to the 14mers are separated and used to regenerate double stranded DNA TAGs, which are then processed, concatenated and sequenced. Since hybridization generally occurs with a 1:1 stoichiometry between a nucleic acid species and a complementary random oligonucleotide, the concentration of each nucleic acid species bound to the random oligonucleotides should be no greater than the concentration of each random oligonucleotide species. Since each species of randomized 14mer is readily controlled and preferably equal across all 14mer sequences, the hybridization step acts to normalize the abundance of nucleic acid species towards the concentration of the randomized 14mers. The concentration of random oligonucleotides is preferably selected such that there will be relatively few copies of each random sequence species relative to the number of copies of higher abundance RNA or cDNA species (thus placing a cap on the abundance of each RNA or cDNA species after normalization). If a more stringent normalization is desirable, the concentration of random oligonucleotides may be selected such that there will be relatively few copies of each sequence even relative to RNA or cDNA species of normal or low abundance. The appropriate concentration of random oligomers may be determined empirically, depending on the desired results. The example shown in FIG. 20 may be further generalized. The TAGs (or other subject nucleic acids) need not be transcribed into single stranded RNA; instead, the nucleic acids may simply be denatured and applied to the random oligomers, or the nucleic acids may be used as templates for single strand PCR to provide single stranded DNA. The random oligomers need not be affixed to a substrate. Instead, the random oligomers may include an affinity purification label that facilitates capture of such random oligomers and any nucleic acids hybridized thereto. Although random oligonucleotides of length 14 are a preferred embodiment, other lengths may be used, such as 7, 9, 10, 11, 12, 13, 14, 15, 16 or more nucleotides. In general, shorter random oligonucleotides will be less specific, while longer random oligonucleotides will result in a larger, more complex pool of oligonucleotides. Nucleic acid species hybridized to the random oligonucleotides may be recovered by a variety of methods such as, for example, by reversing the hybridization (e.g. high temperature or urea) or by selectively degrading the random oligonucleotides. This normalization approach may be used in the context of a TAG preparation protocol, as in the 5′-RED and 5′-3′-RED protocols described herein and the SAGE protocol described elsewhere, and this approach may also be used with longer nucleic acids, e.g., in the normalization of cDNAs or RNAs prior to cDNA library generation.

In certain embodiments, sequences identified as being at or near the 5′-end of a transcript may be used to design probes for use in RNAi procedures. For use in mammalian cells, RNAi probes are generally double stranded RNAs of between 15 and 40 nucleotides in length, although RNAi probes may also be single stranded nucleotides that form hairpin structures. In addition, RNAi probes may be RNA:DNA hybrids, where the antisense strand is RNA. In further embodiments, RNAi probes may contain one or more modified nucleic acids, such as nucleic acids that are modified on the nucleoside ring or in the sugar-phosphate backbone. Phosphorothioate modification in the sugar-phosphate backbone are commonly used.

7. 5′-3′-Co-RNA End Determination

In certain aspects, the disclosure provides methods for determining a sequence from at or near the 5′-end of an RNA and at or near the 3′-end of the same RNA. In certain preferred embodiments, the RNA or cDNA derived therefrom is treated such that sequence at or near the 3′-end tends to be sequence from upstream of the poly-A tail. In general, the poly-A tail is not encoded by genomic sequence, but is added post-transcriptionally by an enzyme. Accordingly, it is preferable to obtain 3′ sequence that is encoded by the genome and can therefore be matched back to a genomic sequence.

In certain embodiments, the disclosure provides methods whereby TAGs from the 5′ end and near 3′ end of a single RNA can be extracted at the same time and located in physical proximity within a nucleic acid. In certain embodiments, a method of the disclosure employs a 5′-labeled-chimeric cDNA generated according to any of the methods described herein or other methods that are, in view of this specification, available to one of skill in the art. In certain embodiments, the poly-A tail or other 3′-portions of the 5′-labeled cDNA may be removed. For example, the 5′-end of the 5′-labeled chimeric cDNA may comprise a recognition site for a first tagging cleavage reagent. The 5′-end of the 5′-labeled-chimeric cDNA may be protected, for example by binding, e.g. via an affinity purification label, to a capture medium, while the 3′-end is exposed to digestion with a nuclease to remove poly-A sequence. In preferred embodiments, the nuclease is nonprocessive, meaning that it is possible to obtain cDNA products that have been partially but not completely digested. Exonuclease III is an example of a nonprocessive enzyme. In certain embodiments, the nuclease may leave single-stranded DNA that may be removed by treatment with a single strand specific nuclease, such as Mung bean or S1 nucleases (Methods in Enzymology, volume 152, 94-110). An adaptor may be attached to the digested 3′-end, wherein the adaptor comprises a recognition site for a second tagging cleavage reagent that is positioned so as to cleave in the 3′-direction from the recognition site. After adapter attachment, the cDNA may be referred to as a 5′-3′-labeled chimeric cDNA. The 5′-3′-labeled chimeric cDNA, if attached to a capture medium may be released by, for example, digestion with a cleavage reagent that cleaves at a site close to the 5′-end (termed the cleavage reagent 1) or by competitive elution with free affinity purification label. The 3′-adaptor may be designed to comprise a recognition site for cleavage reagent I also, and cleavage with that reagent would then create 5′ and 3′-ends that are compatible for ligation. Optionally the ends are complementary single-stranded ends (i.e. “sticky ends”).

As another example of how to eliminate the poly-A tail portion of a 5′-labeled chimeric cDNA, the cDNA is synthesized in a mixture comprising phosphorothioate derivatives of cytosine and guanosine, but standard forms of adenosine and thymidine (dATP, dTTP, dGTP-aS, and dCTP-aS). Accordingly, the poly-A tail portion of the 5′-labeled chimeric cDNA contains little or no phosphorothioate linkages, while the remaining portion (the more 5′-portion) of the 5′-labeled chimeric cDNA comprises phosphorothioate linkages at a frequency proportional to the presence of G and C. Phosporothioate linkages are resistant to certain nucleases, such as exonuclease III (Putney et al. (1981) Proc. Natl. Acad. Sci. USA 78, 7350-7354), and accordingly, a phosphorothioate analog 5′-labeled chimeric cDNA produced as described above may be treated with exonuclease III to remove the poly-A tail, and the exonuclease will be blocked when it encounters a phosphorothioate linkage. Other nucleotide analogs that are resistant to cleavage by an exonuclease may be substituted for the phosphorothioate analogs. The digested nucleic acids may then be blunted and ligated to a 3′-adapter as described above.

To obtain 5′-3′-TAGs comprising a 3′-TAG and a 5′-TAG, the 5′-labeled-chimeric cDNA is circularized, such that the 3′-end becomes attached to the 5′-end. Circularization may be achieved, for example, by an intramolecular ligation reaction, which can be done simply by diluting DNA concentration during ligation reaction to favor intramolecular reactions over intermolecular ligations. This method for circularization has been shown to for nucleic acids between 0.3 to 45 kb DNA. It has been shown that at diluted conditions, near 100% of DNA molecules can undergo intramolecular ligation (PNAS, 1984, 81, 6812-6816). After circularization, the cDNA is cleaved with both the first and second tagging cleavage reagents (although these may be the same, meaning that cleavage would be accomplished at both recognition sites by treatment with a single cleavage reagent) to release a nucleic acid comprising 5′- and 3′-portions of the cDNA, but lacking the middle portion. This nucleic acid may be re-circularized (blunting with, e.g., T4 polymerase may be useful) and amplified using primers that hybridize to the 5′-chimeric portion and the 3′-adaptor portion. The amplified product should contain recognition sites for first and second anchoring cleavage reagents, and these may be present originally in the adaptor and/or chimeric portions or they may be introduced during amplification. The amplified product may then be digested with the first and second cleavage reagents to release 5′-3′-TAGs, comprising a TAG sequence corresponding to a 3′-portion of the acceptor RNA adjacent to a TAG sequence corresponding to a 5′-portion of the acceptor RNA. In certain embodiments, the 3′-TAG sequence and 5′-TAG sequence are arranged such that their 3′-ends (as measured according to the sense-strand) are adjacent (tail-to-tail). In certain embodiments, the 5′-3′-TAGs may be concatenated to form concatemers. In certain embodiments, a concatemer comprises one or more iterated units, wherein an iterated unit comprises, in order, a first cleavage reagent recognition site, a first 5′-3′-TAG, a second cleavage reagent recognition site and a second 5′-3′-TAG.

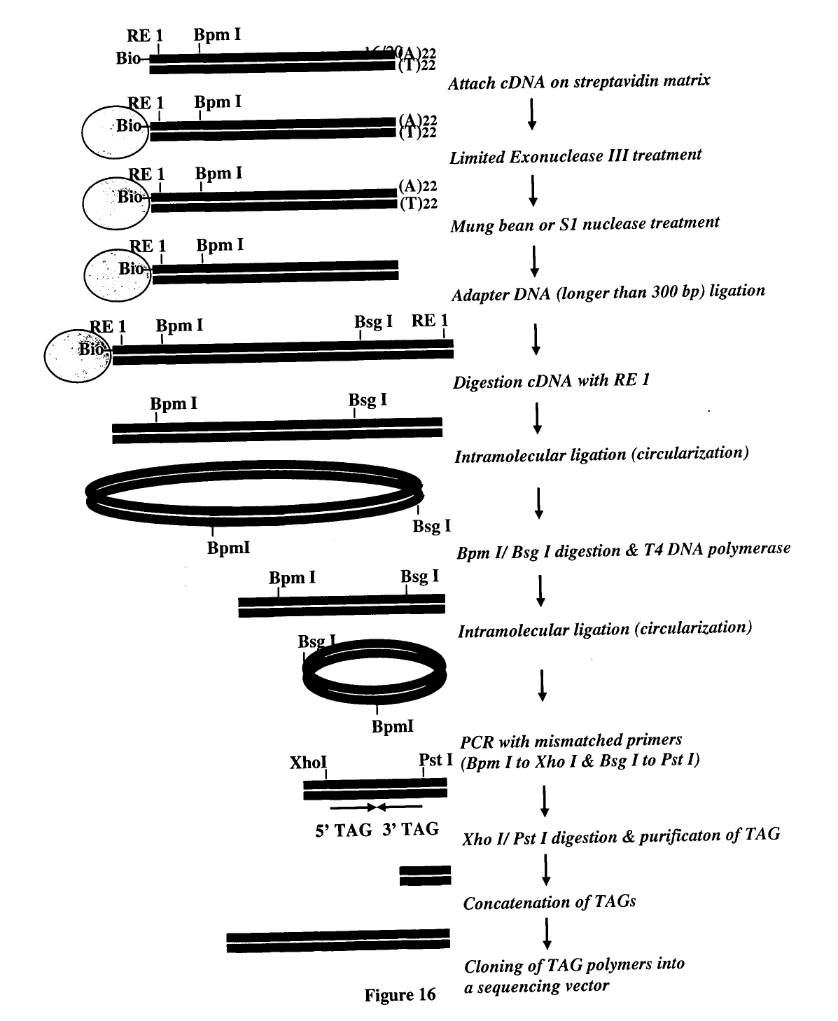

An example, provided in FIG. 16, illustrates a method using Bpm I and Bsg I as first and second tagging cleavage reagents, but other cleavage reagents, including type IIs and III restrictions enzymes can also be used. The RE (restriction enzyme) 1 can be any cleavage reagent, but preferred cleavage reagents are those that have a recognition site of 8 bp or longer, to minimize random cutting the sequence of the cDNA corresponding to the acceptor RNA. Exemplary restriction enzymes with rare recognition sites include Not 1, Fse I, and Asc I.

5′-3′-TAG sequences, however obtained, may then be matched to a genomic sequence. In certain aspects, the disclosure provides methods for identifying, in a genome of a reference organism (the “reference genome”), a genomic region that is likely to encode a transcript that is an ortholog of a transcript of a test organism. Optionally the test organism is an organism for which there is incomplete or insignificant genomic sequence available. In certain embodiments, there is no genomic sequence available for the test organism. Generally the reference organism is one for which a significant amount of genomic sequence is available, and preferably a complete or near-complete genome sequence is available for the reference organism. In certain embodiments, the method comprises accessing a 5′-TAG sequence and a 3′-TAG sequence derived from the transcript of the test organism. The 5′-TAG and 3′-TAG sequences may be compared to the reference genome to identify close or identical matches, termed orthologous genomic sequences. Optionally, an orthologous genomic sequence shares 80% or greater sequence identity with the corresponding TAG sequence, and preferably 85%, 90%, 95% or greater sequence identity. In particularly preferred embodiments, the orthologous genomic sequence is 100% identical to the corresponding TAG sequence. If the method is performed accepting greater dissimilarities between a TAG and a sequence in the reference genome, the identification of the correct or “true” ortholog may be more difficult. If the method is performed accepting few to no dissimilarities between a TAG sequence and a sequence in a reference genome, the likelihood of finding any ortholog may be decreased. Optionally, the matching process may be repeated with several different levels of stringency. A genomic region comprising a 5′-orthologous genomic sequence and a 3′-orthologous sequence in an appropriate orientation is a genomic region that is likely to encode a transcript corresponding to the transcript of the test organism. By appropriate orientation is meant that the 5′- and 3′-orthologous sequences are oriented in the genome with respect to each other in a manner that is consistent with both sequences being transcribed as part of a single transcript. The size of a “genomic region” may be chosen according to the characteristics of gene organization in the reference organism. For example, in a reference organism with a significant number of identified genes, it is possible to generate a distribution of the size of the genomic region occupied by each gene. A genomic region size cut-off for the present analysis may be selected such that greater than 80% of known and/or predicted genes in the reference genome are contained in regions equal to or smaller than the genomic region size cut-off. More stringent cut-offs may be used, such that, for example, 85%, 90%, or 95% or more of the known and/or predicted genes in the reference genome are contained in regions equal to or smaller than the genomic region size cut-off.

8. Probe Arrays

In certain aspects, the disclosure provides probe arrays, and methods for making probe arrays. In certain embodiments, sequences identified as being at or near the 5′-end of a transcript may be used to design arrays of oligonucleotide probes (e.g. DNA “chips”) that detect the presence of the transcript. In situations where the 5′-end sequence distinguishes different transcripts from the same gene, the array will be able to distinguish the various RNA transcripts from a single gene. In certain embodiments, the disclosure provides probe arrays comprising a plurality of oligonucleotide probes corresponding to the 5′-ends of transcripts. In certain embodiments, the disclosure provides probe arrays comprising a plurality of oligonucleotide probes corresponding to 5′-ends and 3′-ends of transcripts. In certain embodiments, the probe array comprises oligonucleotide probes corresponding to at least 100 5′-ends of transcripts, and preferably at least 200, 500, 1000 or at least 5000 5′-ends of transcripts. A probe array may additionally comprise probes that do not correspond to the 5′-ends of transcripts.

In certain embodiments, the disclosure provides methods for preparing probe arrays. For example, RNA may be subjected to 5′RED according to TAG-based methods described herein, and the TAG sequences obtained may be used to make probes for a probe array. In certain embodiments, a probe array may be generated from TAGs derived from an organism for which genome sequence is not known, and therefore the disclosure provides methods for genome scale analysis in organisms for which there is not yet substantial genome sequence information. A probe array may also be used to identify TAGs. Instead of, or in addition to, sequencing TAGs derived from an RNA preparation, TAGs may be contacted with a probe arrays based on TAG sequences from the same organism or a closely related organism, and the identity of TAGs and corresponding transcripts inferred from hybridization information.

Probe arrays generally comprise a surface to which probes that correspond in sequence to gene products (e.g., cDNAs, mRNAs, oligonucleotides) are bound at known positions. In one embodiment, a probe array is an array (i.e., a matrix) in which each position represents a discrete binding site for an RNA (or corresponding cDNA) encoded by a gene and in which binding sites are preferably present for transcripts of most or almost all of the genes in the organism's genome. In a preferred embodiment, the “binding site” (hereinafter, “site”) is a nucleic acid or nucleic acid analogue to which a particular cognate cDNA can specifically hybridize. The nucleic acid or analogue of the binding site can be, e.g., a synthetic oligomer.

Although in a preferred embodiment the microarray contains binding sites for products of all or almost all genes in the target organism's genome, such comprehensiveness is not necessarily required. Usually the microarray will have binding sites corresponding to at least 100 genes and more preferably, 500, 1000, 4000 or more. In certain embodiments, the most preferred arrays will have about 98-100% of the genes of a particular organism represented. In other embodiments, the disclosure provides customized microarrays that have binding sites corresponding to fewer, specifically selected genes. Microarrays with fewer binding sites are cheaper, smaller and easier to produce.

The probes to be affixed to the arrays are typically polynucleotides. These DNAs may be obtained by, e.g., sequencing of TAG sequences as described herein. Oligonucleotides may be chemically synthesized by a variety of well known methods. Synthetic sequences are between about 15 and about 500 bases in length, more typically between about 20 and about 50 bases. In some embodiments, synthetic nucleic acids include non-natural bases, e.g., inosine. As noted above, nucleic acid analogues may be used as binding sites for hybridization. An example of a suitable nucleic acid analogue is peptide nucleic acid (see, e.g., Egholm et al., 1993, PNA hybridizes to complementary oligonucleotides obeying the Watson-Crick hydrogen-bonding rules, Nature 365:566-568; see also U.S. Pat. No. 5,539,083).

The nucleic acids or analogues may be attached to a solid support, which may be made from glass, plastic (e.g., polypropylene, nylon), polyacrylamide, nitrocellulose, or other materials. A method for attaching the nucleic acids to a surface is by printing on glass plates, as is described generally by Schena et al., 1995, Science 270:467-470. This method is especially useful for preparing microarrays of cDNA. (See also DeRisi et al., 1996, Nature Genetics 14:457-460; Shalon et al., 1996, Genome Res. 6:639-645; and Schena et al., 1995, Proc. Natl. Acad. Sci. USA 93:10539-11286).

Techniques are also known for producing arrays containing thousands of oligonucleotides complementary to defined sequences, at defined locations on a surface using photolithographic techniques for synthesis in situ (see, Fodor et al., 1991, Science 251:767-773; Pease et al., 1994, Proc. Natl. Acad. Sci. USA 91:5022-5026; Lockhart et al., 1996, Nature Biotech 14:1675; U.S. Pat. Nos. 5,578,832; 5,556,752; and 5,510,270, each of which is incorporated by reference in its entirety for all purposes) or other methods for rapid synthesis and deposition of defined oligonucleotides (Blanchard et al., 1996, 11: 687-90). When these methods are used, oligonucleotides of known sequence are synthesized directly on a surface such as a derivatized glass slide. In certain embodiments, the array produced is redundant, with several oligonucleotide molecules per RNA. Oligonucleotide probes based on TAG sequences may be chosen to detect alternatively spliced mRNAs or transcripts resulting from alternative transcription start sites.

Other methods for making probe arrays, e.g., by masking (Maskos and Southern, 1992, Nuc. Acids Res. 20:1679-1684), may also be used. In principal, any type of array, for example, dot blots on a nylon hybridization membrane (see Sambrook et al., Molecular Cloning—A Laboratory Manual (2nd Ed.), Vol. 1-3, Cold Spring Harbor Laboratory, Cold Spring Harbor, N.Y., 1989, which is incorporated in its entirety for all purposes), could be used, although, as will be recognized by those of skill in the art, very small arrays will be preferred because hybridization volumes will be smaller.

Arrays preferably include control and reference probes. Control probes are nucleic acids which serve to indicate that the hybridization was effective. Reference probes allow the normalization of results from one experiment to another, and to compare multiple experiments on a quantitative level. Reference probes are typically chosen to correspond to genes that are expressed at a relatively constant level across different cell types and/or across different culture conditions. Exemplary reference nucleic acids include housekeeping genes of known expression levels, e.g., GAPDH, hexokinase and actin.

Mismatch controls may also be provided for the probes to the target genes, for expression level controls or for normalization controls. Mismatch controls are oligonucleotide probes or other nucleic acid probes identical to their corresponding test or control probes except for the presence of one or more mismatched bases.

Exemplary techniques for constructing arrays and methods of using these arrays are described in EP No. 0 799 897; PCT No. WO 97/29212; PCT No. WO 97/27317; EP No. 0 785 280; PCT No. WO 97/02357; U.S. Pat. No. 5,593,839; U.S. Pat. No. 5,578,832; EP No. 0 728 520; U.S. Pat. No. 5,599,695; EP No. 0 721 016; U.S. Pat. No. 5,556,752; PCT No. WO 95/22058; U.S. Pat. No. 5,631,734; U.S. Pat. No. 6,083,697; and U.S. Pat. No. 6,051,380.

9. Protein Labeling

In further aspects, the disclosure provides methods for producing labeled fusion proteins. In certain embodiments, the method comprises expressing in a cell a 5′-trans-splicing nucleic acid that comprises an exon encoding a polypeptide label. The exon is transferred to one or more RNA molecules in the cell, creating 5′-chimeric RNAs encoding fusion proteins that comprise the polypeptide label at the amino-terminus. A polypeptide label may be, for example, a detection label that facilitates detection of a fusion protein, or a purification label, that facilitates purification of a fusion protein. Examples of detection labels include fluorescent proteins (e.g. Green Fluorescent Protein and the many variants thereof), enzymes that catalyze the production of fluorogenic or chromogenic products (e.g. beta-galactosidase, beta-glucuronidase), enzymes that catalyze the destruction of fluorogenic or chromogenic products and epitope tags (short amino acid sequences that are specifically recognized by established monoclonal antibodies, including a myc tag, FLAG tag or VSV tag). Examples of purification labels include polyhistidines (especially hexahistidine sequences, glutathione-S-transferase, thioredoxin, chitin-binding protein, cellulose binding protein and epitope tags (as described above). A polypeptide label may be both a detection label and a purification label.

In certain embodiments, the fusion proteins are detected in vivo by detecting the label. Fluorescent detection labels are particularly amenable to this approach. Optionally, the fusion proteins are detected in vitro by preparing a cell fraction or polypeptide composition and detecting the detection label in the cell fraction or polypeptide composition. In certain embodiments, the 5′-trans-splicing nucleic acid encoding a polypeptide purification label is expressed in a cell specific manner, and proteins in the selected cells are obtained by affinity purification of the purification label. The fusion proteins may then be characterized by a high-throughput method, such as liquid chromatography-mass spectroscopy to obtain a rapid assessment of the protein expression profile in the selected cell types. This approach may allow rapid assessment of the proteomes of different cell types without the need for tedious cell separation techniques.

10. Kits

In certain embodiments, the disclosure provides kits for producing and/or analyzing 5′-chimeric RNAs and cDNAs. The term “kit” as used herein means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. The individual components of the kit may or may not be from the same supplier, or manufacturer. A component can either be purchased as a part of the kit, or generated by user “in-house” according to the instruction of the kit.

In certain embodiments, a kit comprises a 5′-trans-splicing nucleic acid, optionally in a vector for introduction into a cell type of interest. In certain embodiments, the vector is provided as isolated nucleic acid, and in certain embodiments the vector is provided in a carrier cell strain. In certain embodiments, a kit comprises an RNA oligonucleotide for ligation to the 5′-end of RNAs.

In certain embodiments, a kit comprises an oligonucleotide primer. Optionally the oligonucleotide primer is complementary to an exon portion of a 5′-trans-splicing nucleic acid. Optionally, the oligonucleotide is complementary to an RNA oligonucleotide for ligation to the 5′-ends of RNAs. In certain embodiments, an oligonucleotide in the kit comprises an affinity purification label, such as biotin and/or a recognition site for a restriction enzyme.

In certain embodiments a kit comprises a tagging restriction enzyme and/or one or more anchoring restriction enzymes. In certain embodiments a kit comprises one or more enzymes for use in mediating the selective ligation of an RNA oligonucleotide to the 5′-end of capped RNAs. A kit may futher comprise other reagents such as reverse transcriptase, thermostable DNA polymerase, nucleotides, buffers for any of the various enzyme reactions, chromatographic substrates (e.g. oligo dT beads, beads with trans-splicing intron or exon sequence).

In certain aspects, the disclosure provides kits for use in random oligonucleotide-based normalization. A kit for normalization may comprise, for example, a complete set of randomized oligonucleotides. Optionally, the randomized oligonucleotides include an affinity purification label. In certain embodiments, a kit may comprise the set of randomized oligonucleotides that include affinity purification label, and, in addition, a substrate, such as beads or membrane comprising capture reagent. In certain embodiments, the randomized oligonucleotides are provided pre-affixed to a substrate, such as beads or a membrane. Optionally, the substrate is formed into a pre-packed column. The kit may comprise instructions for normalization and may also comprise appropriate hybridization and/or wash buffers. The kit may also comprise adapters and primers for converting nucleic acids into single stranded DNA or RNA. For example, a kit may comprise an adapter having a T7 RNA polymerase promoter site and such a kit may further comprise T7 RNA enzyme or other relevant enzymes, such as thermostable polymerase, RNAse-free DNAse, or DNAse-free RNAse.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### TEC-RED Analysis to Identify 5′-ends of RNA Messages in C. elegans and C. briggsae

TEC-RED was performed successfully in nematode worms to identify the 5′ ends of RNAs. As shown below, this technique permits the identification of alternate transcript initiation sites, previously unknown splice variants, previously unknown 5′-ends of known genes and the 5′-ends of previously unknown genes.

At least 80% of mRNAs in C. elegans and C. briggsae contain SL-1 or SL-2 exons, added by a trans-splicing process. Generally speaking, when pre-mRNAs become mRNAs by cis-splicing and poly-A addition at the 3′-end of the RNA, a SL-1 or SL-2 RNA exon is trans-spliced into the 5′-end of many pre-mRNAs.

In this example of TEC-RED, in brief, the common trans-spliced exon sequence serves as a known sequence at the 5′ end of trans-spliced transcripts that can be used to design primers to direct the synthesis of cDNAs containing the 5′ end of acceptor RNAs. In addition, the primer sequence can be modified to introduce a recognition site for a restriction enzyme with a DNA recognition site that is at a certain distance from the cleavage site. The restriction enzyme treatment extracts a piece of cDNA sequence (generally 14 to 27 bp sequence after the trans-spliced exon) from the 5′-end of each cDNA because its cleavage site is on the cDNA and is 14 to 27 bp apart from its recognition site on the trans-spliced exon. This extracted cDNA piece is termed a ‘TAG’, and the TAG size can be selected to maximize the chance that each TAG is a unique identifier for the RNA from which it derives. If no effort is made to normalize for differences in RNA abundance, an abundant RNA transcript in a RNA mixture will be represented by multiple TAGs, while rarer transcripts will be represented by relatively few TAGs. To facilitate sequencing and identification of TAGS, the TAGs are concatenated and the concatenated TAG polymers are cloned into a plasmid vector for sequencing. TAG sequence data is analyzed using a program that translates TAG sequences into genome sequences to identify the genome sites corresponding to the 5′-RNA ends.

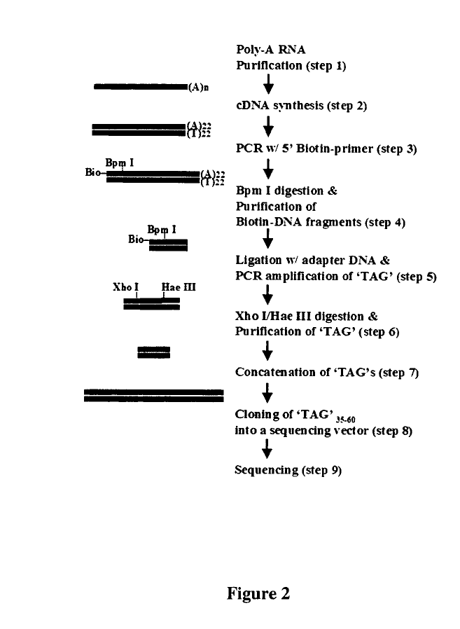

An example of the TEC-RED procedure as performed in C. elegans is diagrammed in FIG. 2. The method starts with the isolation of poly-A RNA (FIG. 2, step 1), which is then used as a template for cDNA synthesis (step 2). The cDNA is amplified by PCR, in which the primer that is complementary to SL-1 sequence contains a biotin moiety and the sequence for a Bpm I recognition site. Amplification with this primer results in the incorporation of biotin at the 5′-end of the cDNA and a Bpm I site at the 3′-end of the SL-1 exon within the cDNA (step 3). Then, the PCR products are digested with Bpm I. Bpm I cleaves the cDNA at a position 14 bp in the 3′ direction from its recognition site. The Bpm I cleavage produces a small cDNA containing a 5′ biotin, followed by SL-1 exon sequence and 14 bp of unknown sequence from the poly-A RNA to which the SL-1 exon was added. The Bpm I enzyme cleavage leaves a 2 bp 3′ overhang, that is treated with T4 DNA polymerase to make blunt ends. The 5′ cDNA pieces are separated from the 3′ pieces by application to streptavidin-magnetic beads, which purifies biotin-labeled DNA fragments (step 4). Biotin-DNA fragment containing the SL-1 exon sequence and the 14 bp TAG are ligated at the 3′ end with an adapter DNA containing a Hae III recognition site. The ligated biotin-DNA fragments are purified from the free adapter DNA, also using streptavidin-magnetic beads and subsequently subjected to PCR amplification with PCR primers containing the Xho I or Hae III recognition site (step 5). The PCR products are purified and digested with Xho I and Hae III. Then, the digested DNA fragments are separated on polyacrylamide gel and the band containing TAGs is eluted from the gel (step 6). The purified TAGs are ligated to form concatemers (step 7). The concatemer containing 35 to 60 TAGs is then purified and ligated into a plasmid vector (step 8). The vectors are transformed into E. coli to be isolated for sequencing (step 9).

In this TEC-RED analysis, several PCR primers are used to extract the TAGs. First, a PCR primer containing a biotin at the 5′-end is used at step 3 of FIG. 2 to provide a basis for affinity purification. Also, base changes within the SL-1 exon sequence (SL-1 sequence TTTGAG is changed to the Bpm I sequence CTGGAG) are introduced to create a Bpm I site at the 3′-end of the same primer. As a result, DNA fragments containing 14 bp TAGs can be obtained by Bpm I treatment. Bpm I is referred to as the “tagging enzyme”. At the next step of PCR amplification process (step 5), this Bpm I recognition site (CTGGAG) is changed to Xho I site (CTCGAG), using a mismatched primer (G to C). Thus, DNA fragments containing the TAGs can be efficiently isolated by digesting with Hae III and Xho I (Step 6). Xho I and Hae III are the “first anchor restriction enzyme” and “second anchor restriction enzyme”, respectively.

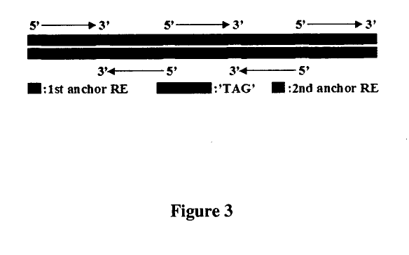

When TAGs are ligated to generate a TAG polymer (concatenation), each TAG is directionally ligated to produce a directional concatemer, as illustrated in FIG. 3. The 5′-end of each TAG is located next to the first anchor restriction enzyme site and the 3′-end is positioned next to the second anchor restriction enzyme site. This kind of directionality is possible, because the DNA cleavage by the first anchor restriction enzyme generates cohesive ends while the cleavage by the second anchor restriction enzyme generates blunt ends.

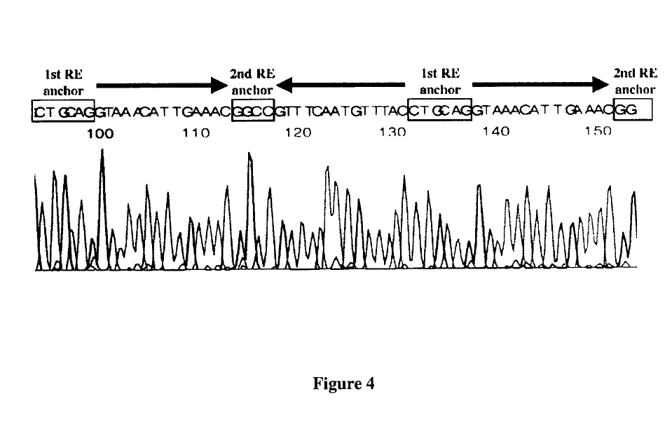

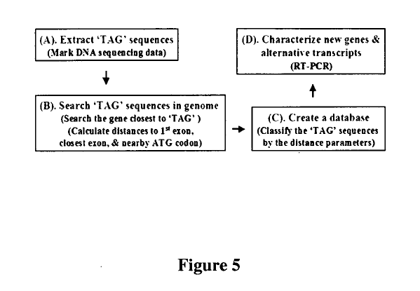

A TEC-RED experiment was performed with C. elegans mRNAs. As shown in FIG. 4, when the RNA was subjected to the TEC-RED analysis using Bpm I/Xho I restriction enzymes, TAGs were identified between the two anchor restriction enzyme sites, and they were directionally positioned with the 5′-end next to the first anchor RE and the 3′-end next to the second anchor RE. Each DNA sequencing reaction was able to identify 30 to 40 TAGs, proving the efficiency of this method for identifying 5′-RNA ends. 45 DNA samples were sequenced by Big-Dye termination method in the presence of dGTP, which greatly improved the DNA sequencing quality. DNA sequencing data was processed as described in the text, following the schemes in FIGS. 5-7.

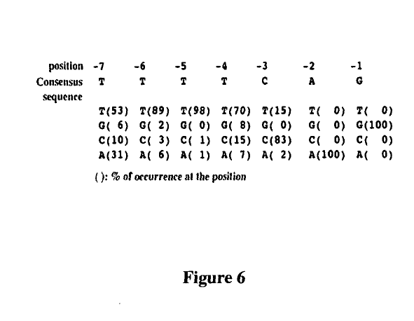

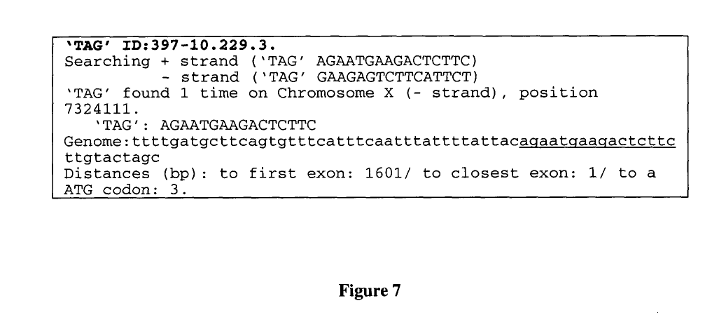

The DNA sequencing data of the concatenated TAG polymers were analyzed as described in FIG. 5. For this analysis, a computer program was developed to locate a TAG within the DNA sequencing data and to use it to search for the matching sites (sequences) within the C. elegans genome sequence. Three facts are considered for this sequence search. First, each TAG sequence should be located after splicing acceptor site (AG) within the genome sequence. Second, the matched genome sites should be oriented in the same direction as the TAG sequence. Third, when a TAG sequence is located more than once within the genome sequence, the matching genome site that follows the conserved splicing acceptor consensus sequence (FIG. 6) is considered as the true corresponding genomic site for the TAG. After identifying the genome site matched with a TAG sequence, the program searches the gene closest to the genome site. Then, the program calculates the distances from the genome site to the first exon, to nearby exon, and to the nearby ATG codon of the gene. These distance parameters are used to judge whether the TAG is located at the known 5′-end or not. First, when the TAG is located at the known 5′-end, the distance to the 1st exon is generally less than 10. Second, when the TAG comes from an additional transcriptional initiation site in an intron of a known gene, the distance from the genome site to the first exon is large, but the distance to the closest exon should be 1. Third, when a TAG is located far from any known gene, both distance parameters become very large. Fourth, when the TAG is located at the new 5′-end of a known gene, both distance parameters are also large, but are smaller than those for unknown genes. At the next stage of analysis, the program classifies the TAGs based on these distance parameters. FIG. 7 shows an example of searching for a TAG sequence in C. elegans genome, representing an alternative transcript created by the second transcriptional initiation. To make sure the TAG analysis program identifies the correct genome sites, each TAG sequence was also individually inspected using short oligo search programs in NCBI and WormBase. For the candidates of new genes and new 5′-ends of known genes were also individually inspected using gene prediction data in each cosmid sequence at NCBI and by WormBase Genome search programs.

Table 2 summarizes results from a TEC-RED analysis. A total 1323 TAGs from C. elegans mRNAs containing SL-1 exon were obtained, and they represent 493 different TAG sequences. 97% of these TAG sequences were found in the C. elegans genome sequence, and most of them correspond to single sequences (98.5%), proving the fidelity of this method. In this analysis, 21 new genes and 7 new 5′-ends of the known genes (about 5% of the TAG sequences) were discovered. Their presence was verified by RT-PCR analysis.

TABLE 2
Summary of a TEC-RED Analysis of C. elegans mRNA
Containing SL-1 Exon.

| | No of |
|---|---|
| Classification of TAG | TAGs |
| Number of obtained TAGs | 1323 |
| Number of TAG sequences | 493 |
| Number of TAG sequences found in C. elegans genome | 480 |
| sequence | |
| Single hit | 473 |
| More than single hit | 7 |
| Number of TAG sequences located in known 5′-ends | 429 |
| Number of TAG sequences indicating unpredicted genes | 21 (15)* |
| Number of TAG sequences indicating unpredicted 5′-ends | 7 (6)* |
| of known genes | |
| Number of TAG sequences indicating 2^{nd} transcription | 22 (20)* |
| start site | |

*() indicates number of cases confirmed by RT-PCR.

### Example 2

### Cell-Based Trans-Splicing Reaction with a Modified SL-1 RNA (mSL-1)

The intron sequence of the C. elegans SL-1 RNA is involved in proper trans-splicing reactions, but partial deletions and site-directed mutagenesis of exon sequence do not significantly perturb the trans-splicing reaction. As demonstrated here, even highly modified sequences of the SL-1 exon could perform trans-splicing properly. A modified SL-1 exon could be used, for example, to introduce a tagging restriction enzyme site that produces tags larger than 14 bp or to introduce leader sequence encoding a useful polypeptide tag.

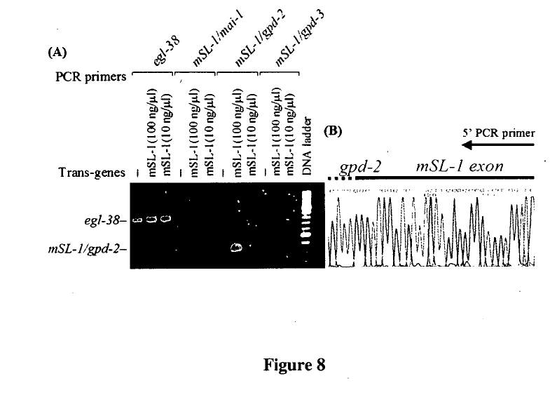

The modified SL-1 (mSL-1) RNA in C. elegans was expressed by an extrachromosomal array. This mSL-1 RNA contains a modified exon sequence (50 bp long sequence different from the original 22 bp long exon sequence) and the original intron sequence of SL-1 RNA. To test whether this mSL-1 RNA could trans-splice specifically to SL-1 acceptable mRNAs, the occurrence of in vivo trans-splicing reactions was assessed for the three genes in mai-1 operon: mai-1, gpd-2, and gpd-3. Among these genes, only gpd-2 undergoes the trans-splicing with the SL-1 RNA. When the total RNA from worms expressing mSL-1 RNA was analyzed by RT-PCR, only the RT-PCR reaction using the 5′-primer of the first half of the mSL-1 exon and the 3′-primer specific to gpd-2 amplified the DNA of expected size (FIG. 8A), indicating that the mSL-1 RNA undergoes trans-splicing with specificity equivalent to that of native SL-1. Sequencing of the amplified DNA showed gpd-2 RNA fused with mSL-1 exon (FIG. 8B). The sequence contains the second half of the mSL-1 exon sequence The sequence analysis showed that the fusion occurred at the expected splicing donor-acceptor sites, proving the trans-splicing reaction occurred as expected between mSL-1 RNA and gpd-2 RNA.

### Example 3

### Separation of mSL-1 RNA and Trans-Spliced RNAs by Exon Affinity Chromatography

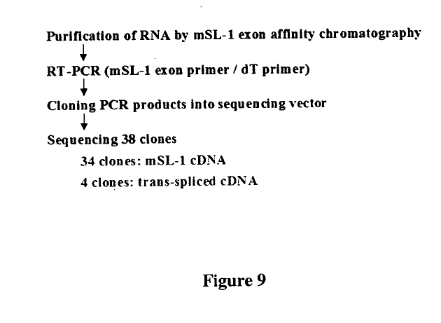

As an additional or alternate method for obtaining RNAs having a trans-splicing exon sequence, a system for affinity purification was developed, based on the sequence of the trans-splicing exon. As shown in FIG. 9, RNA transcripts containing mSL-1 exon at their 5′-end were purified by affinity chromatography, using a column containing oligonucleotide sequences that are complementary to the mSL-1 exon sequence. The purified RNA was subsequently amplified by RT-PCR, and the products were sequenced, following their cloning into a sequencing vector. The sequencing results showed that 90% of the purified transcripts were mSL-1 RNA and the other 10% represented the trans-spliced mRNAs. This technique may be used as a primary technique for isolating RNAs, or in combination with a technique such as oligo-dT isolation of poly-A RNAs. In addition, this technique may be used after RNAs have been converted to cDNAs. This technique may also be used to isolate labeled RNAs from complex biological sources (e.g. mixed cell cultures, tissues, whole organisms) where a trans-splicing nucleic acid has been expressed in a cell-specific manner. This permits a single step separation of RNA from cells of interest from RNA of cells that are not the subject of inquiry.

### Example 4

### Modifications to the Basic TEC-RED Technique

TEC-RED methods can be modified depending on the size of genome to study. TEC-RED methods can use a number of different cleavage reagents. For example three different type IIs restriction enzymes (Bpm I, Bsg I, and Mme I) or a type III (EcoP15I) restriction enzyme may be used, depending on genome size. A larger genome is preferably analyzed using a tagging cleavage reagent that can generate a longer TAG. But, generally the TAG size should be kept as small as is workable with the subject genome because small TAGs decreasing the overall sequencing effort required. Small TAGs (14 to 16 bp) are long enough to analyze C. elegans genome (108 bp), but the TEC-RED analysis for the human genome, which is about 30-fold larger than that of C. elegans, will benefit from a longer TAG (27 bp or more). Table 3 summarizes some possible combinations of restriction enzymes: Tagging RE (RE used to extract TAG from cDNA, step 4 in FIG. 2), and the first and second anchor restriction enzymes (those with digestion sites flanking each TAG and used to release the TAG from adapter DNA fragments, step 6 in FIG. 1). Table 3 also summarizes the size of each TAG and the average numbers of TAGs in a concatenated TAG polymer. For example, Bpm I (tagging RE)/Xho I (1st anchor RE)/Hae III (2nd anchor RE) combination has been used for the TEC-RED analysis of C. elegans mRNA containing trans-spliced SL-1 exon in studies described above.

TABLE 3
Summary of Examples of Restriction Enzymes used for TEC-RED
and 5′-LM-RED analyses.

| | Bpm I | Bsg I | Mme I | EcoP15I |
|---|---|---|---|---|
| Tagging RE | (CTGGAG) | (GTGCAG) | (TCCGAC) | (CAGCAG) |
| 1^{st} anchor RE | Xho I | Pst I | Sal I | Pst I |
| | (CTCGAG) | (CTGCAG) | (GTCGAC) | (CTGCAG) |
| 2^{nd} anchor RE | Hae III | Hae III | Hae III | SnaB I |
| | (GGCC) | (GGCC) | (GGCC) | (TACGTA) |
| TAG size | 14 or 16 bp | 14 or 16 bp | 18 or 20 bp | 27 bp |
| TAGs per | 37 or 34 | 37 or 34 | 31 or 28 | 22 |
| seq rxn* | | | | |

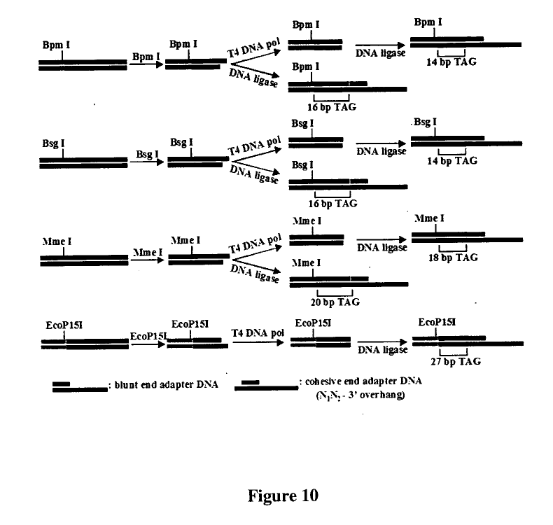

For each combination described above (and for a number of other enzyme combinations, excluding EcoP15 I), two different methods can be used to obtain two different lengths of TAG. These methods are explained in FIG. 10. Here, the DNA fragment cut by Bpm I, Bsg I, or Mme I has a 2 bp-long 3′-overhang structure at the end. After the digestion, two different methods can be applied to generate two different sizes of TAG. In the first approach, the DNA fragment is treated with T4 DNA polymerase to make blunt ends by removing the 2 bp-3′-overhang. Then, the blunted DNA fragment is ligated with an adapter DNA having a blunt end. In the second method, the digested DNA fragments can be directly ligated with an adapter DNA containing cohesive ends. Since any nucleotides can be positioned in the 2 bp-long-3′-overhang, the adapter DNA should have random nucleotides at the first two positions (red adapter in FIG. 10). On the contrary, EcoP15I generates a 5′-overhang DNA end, which is filled by DNA polymerase activity.

### Example 5

### Genome-Scale TEC-RED Analysis to Identify 5′-ends of RNA Transcripts in C. elegans and C. briggsae

For a large-scale application of this TEC-RED analysis to C. elegans and C. briggsae, two additions to the basic TEC-RED analysis may be employed. First, rare RNA transcripts in a RNA mixture are enriched, which facilitates the determination of the 5′-ends of the rare RNA transcripts. This reduces DNA sequencing efforts by reducing the number of duplicate TAGs that will be obtained from highly expressed mRNAs, and fewer total TAGs will need to be sequenced in order to obtain sequences of TAGs from rare transcripts. A subtractive hybridization method, SSH-PCR is applied to normalize RNA mixtures before the TEC-RED analysis. Second, unlike C. elegans, C. briggsae genome sequence has not been extensively annotated. Thus, modifications to the current TAG analysis program may be made to facilitate the search for 5′-ends of genes in C. briggsae.

Additions to TEC-RED that assist with this type of genomic analysis include: normalization of RNA or cDNA abundance, an efficient sequencing strategy, translation of C. elegans TAG sequences into its genome, and translation of C. briggsae TAG sequences into its genome. C. elegans or C. briggsae RNA of different stages is used for this research.

(i). Normalization of RNA abundance: To efficiently identify rare transcripts, the concentration of high- and low-abundant sequences in a cDNA mixture is equalized by a subtractive hybridization method, SSH-PCR. This normalization works because re-annealing is faster for the more abundant molecules due to the second-order kinetics of hybridization. Currently available SSH-PCR methods are designed to effect subtraction of whole restriction enzyme-digested cDNA fragments, and this would remove alternative transcripts having, for example, different 5′ ends but largely similar 3′ sequences from the analysis. Thus, current protocol of SSH-PCR will be modified to preserve the advantage of TEC-RED method, detecting 5′-ends of alternative transcripts from a single gene. As illustrated in detail (FIG. 11), in this modified protocol, the 5′-cDNA fragments are obtained by the procedures in box (A) and applied to SSH-PCR (box B in FIG. 11). Portions of the SSH-PCR are carried out as described in a protocol provided by CLONTECH (Foster City, Calif.). After SSH-PCR, the normalized 5′-cDNA fragments contain a Bpm I (a tagging restriction enzyme, FIG. 2 and Table 2) site and biotin group at their 5′-end. These cDNA fragments can be directly introduced to TEC-RED analysis (to step 4 in FIG. 2).

(ii). DNA sequencing plan: The standard and the normalized cDNA mixtures are applied to TEC-RED analysis in order to detect abundant and rare RNA transcripts. Automated plasmid mini-preparations provide DNA templates for the DNA sequencing with capillary electrophoresis (ABI 3700 model). The study described above showed that 1323 TAGs identified 480 different TAG sequences (36%). Thus, 2400 DNA sequencing reactions sequence about 84,000 TAGs and, thus, identify about 30,000 different TAG sequences. Since the normalized cDNA mixtures are used, more than 30,000 different TAG sequences are expected.

There are 20,000 predicted genes in C. elegans. Thus, these more than 30,000 TAG sequences are enough to identify 5′-ends of most genes and their alternative transcripts having different 5′-ends in C. elegans.

(iii). Translation of TAG sequence into C. elegans genome sequence: The strategy used to analyze the TEC-RED analysis on C. elegans RNA may be used to analyze this large-scale data.

(iv). Translation of TAG sequence into C. briggsae genome sequence: C. briggsae genome sequence has been assembled without gene annotation. Although its full annotation is expected in the near future, it may be useful to have a strategy to identify genes corresponding to the TAG sequences in this genome before its full gene annotation. First, the site for each TAG sequence is searched in the C. briggsae genome sequence using the same program used for C. elegans genome. Second, genomic sequences (about 1-2 kb) next to the corresponding genomic sites of TAG sequences are blasted against C. elegans genome sequence. Since the exon sequences in both nematodes are known to be highly similar, this Blast search finds orthologs (high similarity in exon sequences) in many cases. There should be some C. briggsae genome sites that do not correspond to any sequences in C. elegans genome, which may represent distinct genes in C. briggsae from those in C. elegans. Further bioinformatics may be used to obtain additional characteristics of these genes.

### Example 6

### Cell-Specific Expression of mSL-1

To label the 5′-ends of RNA transcripts expressed in specific cells of C. elegans, constructs with four different cell-specific enhancers were prepared, driving the expression of mSL-1 RNA in C. elegans neuronal cells (aex-3 enhancer element) (Iwasaki et al. 1997), muscle cells (unc-54 enhancer element) (Waterston et al. 1982), vulval cells/male-specific tail cells (lin-31 enhancer element) (Tan et al. 1998), or a single anchor cell in gonad (lin-3 AC-specific enhancer element), respectively. The expression specificities for these enhancer elements were tested by GFP reporter assay, and cell-specific expression was observed.

The expression of mSL-1 RNA, using a cell-specific enhancer element, will allow labeling RNA transcripts in specific cell types, thus permitting the purification of the transcripts from the given cell by oligonucleotide affinity chromatography. The modified exon region can also be used for creating different restriction enzyme.

### Example 7

### Cell-Specific Transcript Analysis in C. elegans Muscle and Neurons

RNA in C. elegans neuronal or muscle cells that selectively express mSL-1 trans-splicing nucleic acid are purified and linearly amplified, and the amplified RNA is subjected to TEC-RED analysis after the subtraction with SSH-PCR.

Two modifications to the TEC-RED may improve the use of this technique for analysis of cell-specific transcripts. First, a protocol to separate trans-spliced mRNA from the mSL-1 RNA is used. Second, since the labeling happens to only a fraction of RNA in each cell, a method that linearly amplifies a population of RNA transcripts from specific cells is used. In this research, as a model system, trans-spliced RNA from neurons and muscle cells is used, which can be obtained by expressing a modified SL-1 RNA using aex-3 (pan-neuron) and unc-54 (pan-muscle) enhancer elements, as described above.

(i). Preparation of oligonucleotide affinity chromatography matrices: Four different methods are used to couple oligonucleotides onto solid matrices. First, affinity column of oligotex beads (dC10T30 oligonucleotides covalently linked to the surface of polystyrene-latex particles) are used to purify mRNA from total RNA. Second, for a large-scale RNA purification, oligonucleotides containing an amino group (NH2) at their 3′-ends are covalently coupled to N-hyroxysuccinimide (NHS)-agarose (Hammarsten and Chu 1998). This method gives a high coupling efficiency of nucleic acids to the matrix. In some cases, the RNA hybridized with biotinylated oligonucleotides are captured onto streptavidin magnetic beads. When small amounts of RNA is handled, the RNA and biotinylated oligonucleotide hybrids are captured onto a PCR tube coated with streptavidin, which facilitates buffer exchanges during purification. This method is convenient for handling small amounts of RNA or cDNA.

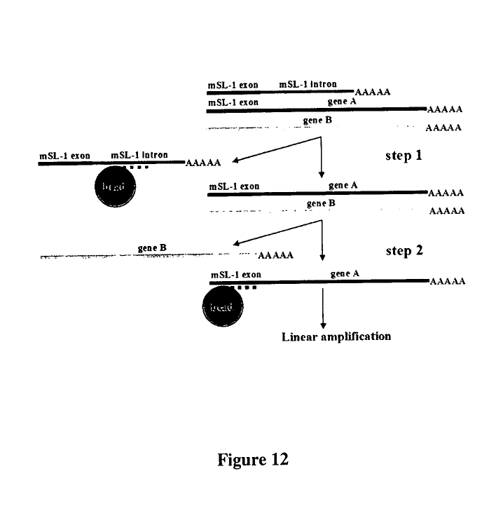

(ii). Affinity purification of 5′-labeled RNA from C. elegans neuronal and muscle cells: In the new purification scheme (FIG. 12), poly-A RNA is subjected to an affinity chromatography containing oligonucleotides complementary to the intron sequence of mSL-1 (step 1). Since free mSL-1 RNA binds to the bead through its intron sequence, the RNA transcripts that do not bind to the affinity column are collected and used for the next step. The second affinity chromatography containing oligonucleotides complementary to the mSL-1 exon sequences purify and enrich the mRNA population tagged with mSL-1 exon by trans-splicing reaction (step 2). Even though these purification steps allow the high enrichment of trans-spliced mRNA over the mSL-1 RNA, purer populations of cell-specific RNA may be obtained by using a linear amplification method discussed immediately below.

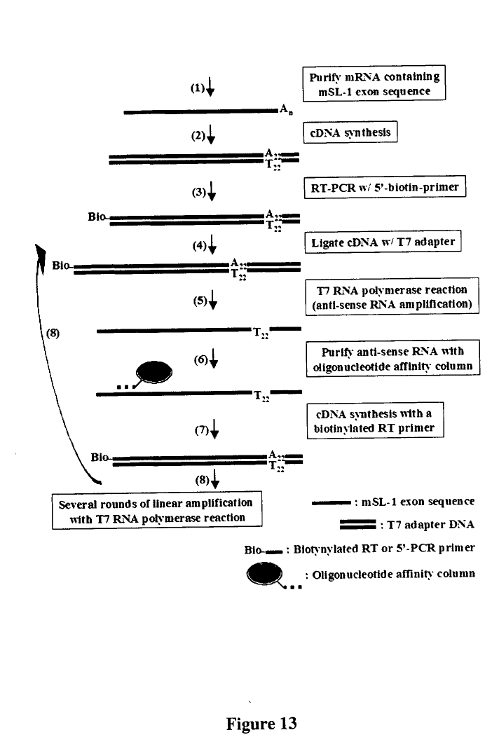

(iii). Linear amplification of full-length cDNA: As illustrated in FIG. 13, a 50 bp mSL-1 exon is used for two different purposes during purification and linear amplification of full-length RNA. The first half of the trans-spliced exon (green bar sequence) is used as a primer for reverse transcriptase (step 7) and RT-PCR (step 3). The second half of the exon (red bar sequence), is used for affinity purification of anti-sense RNA (step 6). Since the sequence for the RT-PCR is different from the sequence used for the affinity purification, the RNA after both amplification and purification processes will represent more pure population of trans-spliced mRNA.

In the linear amplification protocol (FIG. 13), a low number of PCR cycles are applied to the initial amplification step (step 3). This step can be replaced by SP6 RNA polymerase dependent amplification by engineering the polymerase recognition site into the mSL-1 exon or by priming the 2nd strand cDNA synthesis reaction with a primer (green bar) containing an SP6 recognition sequence. Linear amplification of RNA by T7 RNA polymerase reaction has been successfully demonstrated by others (Schena et al. 1995; Phillips and Eberwine 1996; Baugh et al. 2001), and these protocols may be modified to selectively amplify trans-spliced mRNA from C. elegans.

By using a linear amplification technique that generates a single-stranded antisense RNA, it is possible then to perform sequence-specific affinity purification using, as mentioned above, oligonucleotide affinity columns that bind to the antisense of the second half of the mSL-1 exon sequence.

The protocol described in FIG. 13 is designed to linearly amplify full-length RNA. However, it is known that reverse transcriptase can not efficiently synthesize longer transcripts. Thus, when cDNA is synthesized from RNA mixture, small RNA is more efficiently synthesized than longer RNA. This bias against long transcripts may be disadvantageous when gene expression is measured by TEC-RED method that analyzes the 5′-ends. Therefore, the first strand cDNA synthesis by reverse transcriptase may be primed with random oligonucleotide primers instead of oligo (dT) primer when amplified RNA is used for TEC-RED analysis.

(iv). TEC-RED analysis of amplified RNA: To identify preferentially expressed genes in neurons and muscle cells, the purified and linearly amplified neuronal and muscle RNA are subtracted from each other using the SSH-PCR method, before the TEC-RED analysis. This SSH-PCR method carries out normalization and subtraction in a single procedure, which allows more efficient identifications of both abundant and rare transcripts, differentially expressed in two comparing RNA mixtures. In addition, the modified protocol for the subtraction only subtracts 5′-cDNA fragments. Thus, TEC-RED analysis of the subtracted RNA distinguishes alternative transcripts having different 5′-ends from a single gene, preferentially expressed in neurons or muscle cells.

Several modifications of the normalization procedure may be used to apply these amplified RNA to SSH-PCR/TEC-RED analysis. First, since the biotin-labeled amplified cDNA (after step 7 and after several cycles of T7 RNA polymerase reactions in FIG. 13) is used to isolate the 5′-cDNA fragments (box A in FIG. 11), the first PCR step in the box A of FIG. 11 may be omitted. Second, the Xho I and Bpm I combination is replaced with other sets of restriction enzymes such as Pst I and Bsg I (Table 2 and section 4.0.). Third, two different RNA mixtures of neurons and muscle cells are used as Tester and Driver. If desired, RNA from the whole worm cells will be prepared and used as a Driver.

### Example 8

### TEC-RED Analysis to Identify 5′-End of RNA Messages in Human and Mouse

The human and mouse genomes are 30 times larger than the C. elegans genome, and they also have a less conserved splicing acceptor site than nematodes. Thus, it would be helpful to use a tagging restriction enzyme that can generate longer TAG than Bpm I and Bsg I, such as MmeI or EcoP15 I. The Mme I enzyme is available from New England Biolabs (Beverly, Mass.). Alternatively, the enzyme may be purified from Methylophilus methylotrophus by following a previously established purification procedure that includes ion exchange and affinity chromatography. Mme I activity may be assayed by the cleavage of the two Mme I recognition sites on pUC19 DNA. The genes of EcoP15I, which is composed of two subunits, is cloned into E. coli plasmid expression vector (low copy number), and the enzyme is purified from the bacteria expressing the recombinants (Meisel et al. 1995; Mucke et al. 2001). During the cloning, a (His)₆-tag will be added to the amino- or the carboxyl-terminus of each subunit for rapid and efficient affinity purification.

In certain versions, the success of TEC-RED relies on the fact that SL-1 and SL-2 RNA can transfer their exon into different RNA molecules by trans-splicing reaction. Unlike nematodes in which trans-splicing reaction happens naturally to most RNA messages, other organisms may require artificial introduction of trans-splicing reaction to label a common sequence motif at the 5′-end of RNA transcripts. In humans, it has been shown that splicing leader RNAs of C. elegans and trypanosomes can carry out trans-splicing reaction in vivo and in vitro. An mSL-1 RNA that encodes the desired tagging enzyme restriction site may be used. As described above, the exon of SL-1 is easily modifiable.

Any of the other modification described herein may also be used for analysis in humans, mice and other organisms.

### Example 9

### In Vitro Identification of the 5′-Ends of Human RNA Transcripts

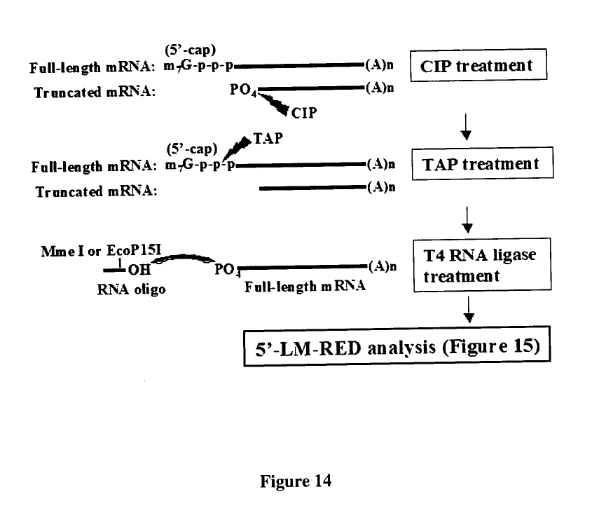

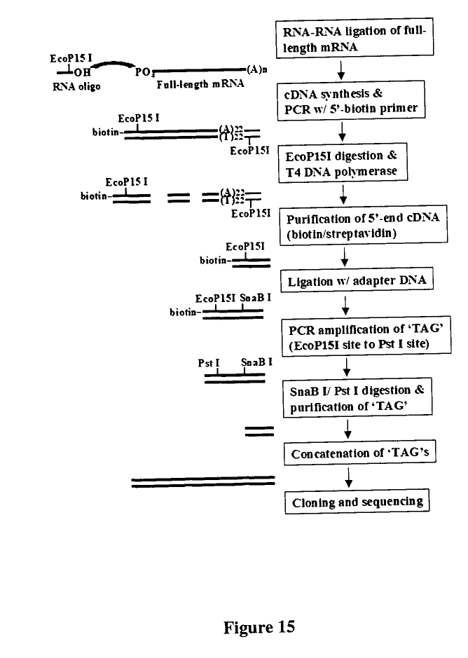

It is possible to perform 5′-RED techniques without using a trans-splicing reaction. An in vitro method termed 5′-ligase mediated RNA end determination (5′-LM-RED) is illustrated in FIGS. 14 and 15. An adapter RNA containing a recognition site for a type IIs or III restriction enzymes is ligated in vitro onto the 5′-ends of the full-length RNA transcripts, but not to the truncated RNA transcripts, by virtue of the endogenous m7-G cap structure on the full-length RNA transcripts (FIG. 14). After this in vitro RNA-RNA ligation step, TAGs are extracted by treating the corresponding restriction enzyme, and the extracted TAGs are concatenated. Then, the concatenated TAG polymers are cloned into a plasmid vector for sequencing, following the same protocol used for the TEC-RED analysis (FIG. 15).

For one-to-one matching between a TAG sequence and its corresponding gene with genome sequence, a longer TAG is preferred for organisms with larger genome size. Three different sizes of TAGs (18, 20, or 27 bp) are readily available with present restriction enzymes, depending on the size of genomes to be analyzed. For example, for human genome with 3×109 bp, 27 bp TAG will be used to achieve the 10⁻¹⁶ probability of having the same TAG sequences more than once within human genome sequence. Consequently, most TAG sequences will correspond to a unique RNA transcript sequence.

To efficiently identify rare RNA transcripts, RNA abundance may be normalized by SSH-PCR following the same protocol for the nematodes. Then, the normalized RNA is subjected to 5′-LM-RED to identify the 5′-ends. For this RED analysis with human RNA, the programs and the analysis schemes used for the analysis of C. elegans and C. briggsae 5′-RNA ends may be applied with minor modifications.

(i). Purification of Mme I and EcoP15I enzymes: Methods for obtaining Mme I and EcoP15I are described above.

(ii). Development of the 5′-LM-RED method: Both TEC-RED and 5′-LM-RED methods share the same steps except for how a common oligonucleotide sequence is added to the 5′-ends of mRNA. In the TEC-RED, in vivo trans-splicing reaction adds a splicing leader exon sequence to mRNA, and, in the 5′-LM-RED, in vitro RNA-RNA ligation selectively adds RNA oligonucleotides to the 5′-ends of full-length mRNA, but not to those of partially degraded mRNA.

As illustrated in FIG. 14, in 5′-LM-RED, purified poly-A RNA is treated sequentially with two phosphatases with different biochemical properties: Calf intestinal phosphatase (CIP) and Tobacco acid pyrophosphatase (TAP). CIP can not remove the 5′-phosphate group protected by a cap structure (m7G-P3). TAP treatment leaves one phosphate group attached to the 5′-end of RNA. Therefore, sequential treatments of these two phosphatases removes the 5′-phosphate group only from truncated mRNA and not from full-length mRNA. As a result, the subsequent T4 RNA ligase treatment selectively attaches RNA oligonucleotides to the 5′-ends of full-length mRNA. Now, this ligated mRNA can undergo the RED analysis following the protocol (FIG. 15) that is slightly modified from TEC-RED protocol in FIG. 2. Only two minor modifications are made from TEC-RED method. First, EcoP15 I is used as a tagging restriction enzyme, which allows longer TAGs (27 bp) to analyze human RNA transcripts. Second, SnaB I is used as the 2nd anchor RE (FIG. 15).

### Example 10

### Ortholog Search Using Sequenced Genomes as References

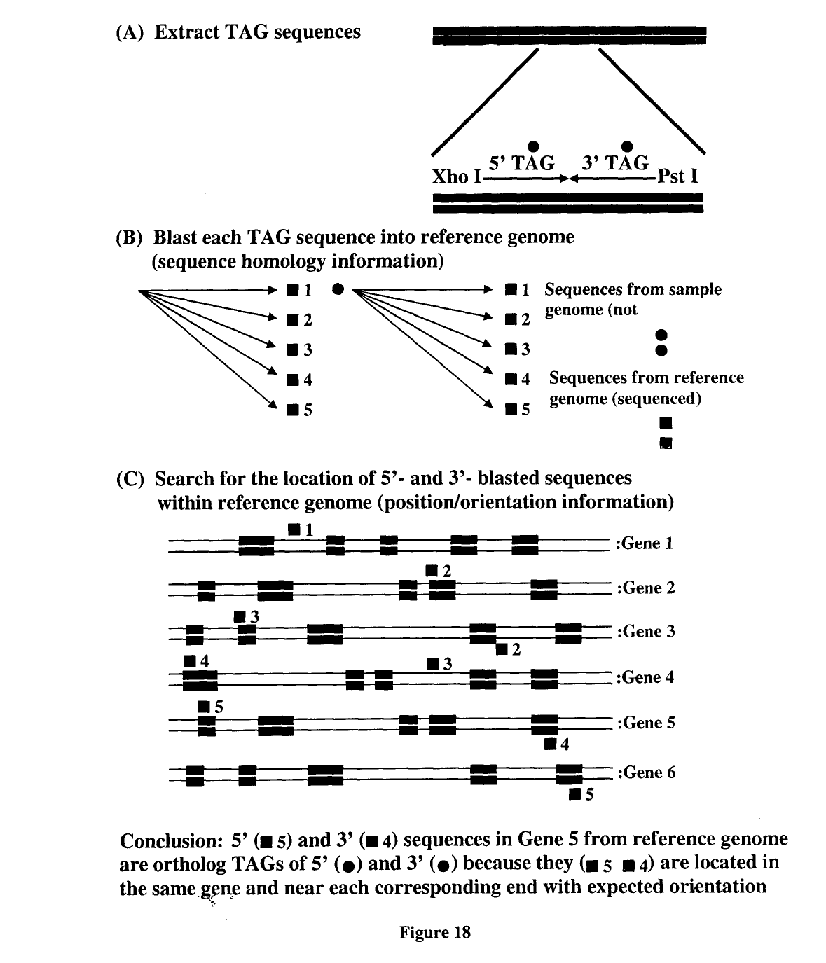

Sequenced genomes may serve as references for functional genomics studies of genomes that have not been sequenced, as diagrammed in FIG. 18. For example, a 5′3′-Co-RED method may be used to obtain the 5′- and 3′-RNA end sequences from an organism for which the genome has not been sequenced (sample or test species) (Panel A), each TAG sequence is compared against a sequenced genome, preferably a genome of a related organism (reference species) (panel B), and BLAST search output sequences (homologous sequences from the reference genomes) are examined for their locations within their own genomes, relative to the location of genes (Panel C). The gene in the reference genome, which contains the corresponding 5′- and 3′-sequences at near each end with the expected orientation, is likely to be the ortholog for the gene in the sample genome whose sequence has not been determined yet. Search results may be experimentally verified by isolating the corresponding cDNA from the sample species, and then comparing the cDNA and predicted amino acid sequences from the sample organism to the gene sequences (also their translated protein sequences from the predicted coding sequences) of potential orthologs in the reference species.

In this approach, information on the 5′- and 3′-TAG sequences from the sample species, and the corresponding 5′- and 3′-sequences from the gene in the reference species, are used for the gene expression analysis using RED methods. This technique pemits gene expression analysis in organisms for which genomes have not been sequenced.

### INCORPORATION BY REFERENCE

All publications and patents mentioned herein, including those listed below, are hereby incorporated by reference in their entirety as if each individual publication or patent was specifically and individually indicated to be incorporated by reference. In case of conflict, the present disclosure, including any definitions herein, will control.

Baugh, L. R., A. A. Hill, E. L. Brown, and C. P. Hunter. 2001. Quantitative analysis of mRNA amplification by in vitro transcription. Nucleic Acids Res 29: E29.

Blumenthal, T. 1998. Gene clusters and polycistronic transcription in eukaryotes.

Bioessays 20: 480-7.

Boyd, A. C., I. G. Charles, J. W. Keyte, and W. J. Brammar. 1986. Isolation and computer-aided characterization of MmeI, a type II restriction endonuclease from Methylophilus methylotrophus. Nucleic Acids Res 14: 5255-74.

Brown, C. T., A. G. Rust, P. J. Clarke, Z. Pan, M. J. Schilstra, T. De Buysscher, G. Griffin, B. J. Wold, R. A. Cameron, E. H. Davidson, and H. Bolouri. 2002. New computational approaches for analysis of cis-regulatory networks. Dev Biol 246: 86-102.

Bruzik, J. P. and T. Maniatis. 1992. Spliced leader RNAs from lower eukaryotes are trans-spliced in mammalian cells. Nature 360: 692-5.

Bruzik. J. P. and T. Maniatis. 1995. Enhancer-dependent interaction between 5′ and 3′ splice sites in trans. Proc Natl Acad Sci USA 92: 7056-9.

Datson, N. A., J. van der Perk-de Jong, M. P. van den Berg, E. R. de Kloet, and E. Vreugdenhil. 1999. MicroSAGE: a modified procedure for serial analysis of gene expression in limited amounts of tissue. Nucleic Acids Res 27: 1300-7.

Diatchenko, L., Y. F. Lau, A. P. Campbell, A. Chenchik, F. Moqadarn, B. Huang, S. Lukyanov, K. Lukyanov, N. Gurskaya, E. D. Sverdlov, and P. D. Siebert. 1996. Suppression subtractive hybridization: a method for generating differentially regulated or tissue-specific cDNA probes and libraries. Proc Natl Acad Sci USA 93: 6025-30.

Eberwine, J. 2001. Single-cell molecular biology. Nat Neurosci 4 Suppl: 1155-6.

Emmert-Buck, M. R., R. F. Bonner, P. D. Smith, R. F. Chuaqui, Z. Zhuang, S. R. Goldstein, R. A. Weiss, and L. A. Liotta. 1996. Laser capture microdissection. Science 274: 998-1001.

Ferguson, K. C., P. J. Heid, and J. H. Rothman. 1996. The SL1 trans-spliced leader RNA performs an essential embryonic function in Caenorhabditis elegans that can also be supplied by SL2 RNA. Genes Dev 10: 1543-56.

Fire, A., S. Xu, M. K Montgomery, S. A. Kostas, S. E. Driver, and C. C. Mello. 1998. Potent and specific genetic interference by double-stranded RNA in Caenorhabditis elegans. Nature 391: 806-11.

Hammarsten, O. and G. Chu. 1998. DNA-dependent protein kinase: DNA binding and activation in the absence of Ku. Proc Natl Acad Sci USA 95: 525-30.

Lockhart, D. J., H. Dong, M. C. Byrne, M. T. Follettie, M. V. Gallo, M. S. Chee, M. Mittmann, C. Wang, M. Kobayashi, H. Horton, and E. L. Brown. 1996. Expression monitoring by hybridization to high-density oligonucleotide arrays. Nat Biotechnol 14: 1675-80.

Luo, L., R. C. Salunga, H. Guo, A. Bittner, K. C. Joy, J. E. Galindo, H. Xiao, K. E. Rogers, J. S. Wan, M. R. Jackson, and M. G. Erlander. 1999. Gene expression profiles of laser-captured adjacent neuronal subtypes. Nat Med 5: 117-22.

Meisel, A., P. Mackeldanz, T. A. Bickle, D. H. Kruger, and C. Schroeder. 1995. Type III restriction endonucleases translocate DNA in a reaction driven by recognition site-specific ATP hydrolysis. Embo J 14: 2958-66.

Mucke, M., S. Reich, E. Moncke-Buchner, M. Reuter, and D. H. Kruger. 2001. DNA cleavage by type III restriction-modification enzyme EcoP15I is independent of spacer distance between two head to head oriented recognition sites. J Mol Biol 312: 687-98.

Nilsen, T. W. 1992. Trans-splicing in protozoa and helminths. Infect Agents Dis 1: 212-8.

Nilsen, T. W. 1993. Trans-splicing of nematode premessenger RNA. Annu Rev Microbiol 47: 413-40.

Phillips, J. and J. H. Eberwine. 1996. Antisense RNA Amplification: A Linear Amplification Method for Analyzing the mRNA Population from Single Living Cells. Methods 10: 283-8.

Reboul, J., P. Vaglio, N. Tzellas, N. Thierry-Mieg, T. Moore, C. Jackson, T. Shin-i, Y. Kohara, D. Thierry-Mieg, J. Thierry-Mieg, H. Lee, J. Hitti, L. Doucette-Stamm, J. L. Hartley, G. F. Temple, M. A. Brasch, J. Vandenhaute, P. E. Lamesch, D. E. Hill, and M. Vidal. 2001. Open-reading-frame sequence tags (OSTs) support the existence of at least 17,300 genes in C. elegans. Nat Genet 27: 332-6.

Schena, M., D. Shalon, R. W. Davis, and P. O. Brown. 1995. Quantitative monitoring of gene expression patterns with a complementary DNA microarray. Science 270: 467-70.

Schwartz, S., Z. Zhang, K. A. Frazer, A. Smit, C. Riemer, J. Bouck, R. Gibbs, R. Hardison, and W. Miller. 2000. PipMaker—a web server for aligning two genomic DNA sequences. Genome Res 10: 577-86.

Southern, E. M., S. C. Case-Green, J. K. Elder, M. Johnson, K. U. Mir, L. Wang, and J. C. Williams. 1994. Arrays of complementary oligonucleotides for analysing the hybridisation behaviour of nucleic acids. Nucleic Acids Res 22: 1368-73.

Stover, N. A. and R. E. Steele. 2001. Trans-spliced leader addition to mRNAs in a cnidarian. Proc Natl Acad Sci USA 98: 5693-8.

Tucholski, J., P. M. Skowron, and A. J. Podhajska. 1995. MmeI, a class-IIS restriction endonuclease: purification and characterization. Gene 157: 87-92.

Tucholski, J., J. W. Zmijewski, and A. J. Podhajska. 1998. Two intertwined methylation activities of the MmeI restriction-modification class-IIS system from Methylophilus methylotrophus. Gene 223: 293-302.

Vandenberghe, A. E., T. H. Meedel, and K. E. Hastings. 2001. mRNA 5′-leader trans-splicing in the chordates. Genes Dev 15: 294-303.

Velculescu, V. E., L. Zhang, B. Vogelstein, and K. W. Kinzler. 1995. Serial analysis of gene expression. Science 270: 484-7.

Webb, B. J., J. S. Liu, and C. E. Lawrence. 2002. BALSA: Bayesian algorithm for local sequence alignment. Nucleic Acids Res 30: 1268-77.

Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method for producing 5′-labeled chimeric cDNA, the method comprising:
a) forming a mixture comprising:
i) a cDNA preparation derived from cells expressing a 5′-trans-splicing nucleic acid, wherein the 5′-trans-splicing nucleic acid comprises an exon and an intron;
ii) an oligonucleotide that has a sequence that hybridizes to at least a portion of the exon of the 5′-trans-splicing nucleic acid, and wherein the oligonucleotide comprises a label; and
iii) an enzyme that catalyzes polynucleotide synthesis; and
b) incubating the mixture under conditions that permit polynucleotide synthesis.

2. The method of claim 1, wherein the cDNA preparation is prepared by a method comprising:
a) obtaining an RNA preparation from the cells expressing the 5′-trans-splicing nucleic acid;
b) synthesizing one or more antisense cDNA strands and optionally synthesizing one or more sense cDNA strands.

3. The method of claim 2, wherein the RNA preparation is enriched for poly-A RNAs.

4. The method of claim 2, wherein the RNA preparation is enriched for RNAs comprising the exon of the 5′-trans-splicing nucleic acid.

5. The method of claim 4, wherein the RNA preparation is enriched for RNAs comprising the exon of the 5′-trans-splicing nucleic acid by a method comprising: contacting the RNA preparation with an exon purification oligonucleotide having a sequence that hybridizes to at least a portion of the exon.

6. The method of claim 2, wherein the RNA preparation is depleted for RNAs comprising the intron of the 5′-trans-splicing nucleic acid.

7. The method of claim 6, wherein the RNA preparation is depleted for RNAs comprising the intron of the 5′-trans-splicing nucleic acid by a method comprising contacting the RNA preparation with an intron purification oligonucleotide having a sequence that hybridizes to at least a portion of the intron.

8. The method of claim 1, wherein the exon comprises a sequence that is at least 80% identical to a sequence selected from the group consisting of SEQ ID NOs: 1, 4, 7 and 8.

9. The method of claim 1, wherein the intron comprises a sequence that is at least 80% identical to a sequence selected from the group consisting of SEQ ID NOs: 2 and 5.

10. The method of claim 1, wherein the oligonucleotide comprises a recognition sequence for a cleavage reagent.

11. The method of claim 10, wherein the cleavage reagent is restriction enzyme.

12. The method of claim 10, wherein the cleavage reagent has a cleavage site that is at least 7 base pairs distant from the recognition site.

13. The method of claim 11, wherein the restriction enzyme is selected from the group consisting of: a type IIs restriction enzyme and a type III restriction enzyme.

14. The method of claim 10, wherein the oligonucleotide comprises a sequence selected from the group consisting of: 5′-CTGGAG-3′,5′-GTGCAG-3′,5′-TCCGAC-3′,5′-CAGCAG-3′.

15. The method of claim 1, wherein the oligonucleotide comprises an attached or incorporated label.

16. The method of claim 15, wherein the attached or incorporated label is a biotin.

17. The method of claim 1, wherein the cells are eukaryotic cells.

18. The method of claim 1, wherein the cells are selected from the group consisting of: cultured cells, cells from a tissue sample, cells from an organism.

19. A method for producing a 5′-labeled chimeric cDNA, the method comprising:
a) obtaining an RNA preparation from cells expressing a 5′-trans-splicing nucleic acid, wherein the 5′-trans-splicing nucleic acid comprises an exon and an intron;
b) synthesizing one or more antisense cDNAs by incubating the RNA preparation in a mixture comprising a downstream primer and a reverse transcriptase;
c) synthesizing a 5′-labeled sense cDNA by incubating one or more of the antisense cDNAs in a mixture comprising an enzyme that catalyzes polynucleotide synthesis and an upstream primer that hybridizes to at least a portion of the exon of the 5′-trans-splicing nucleic acid, wherein the oligonucleotide comprises a label.

20. A method for producing 5′-labeled chimeric RNAs, the method comprising:
expressing in cells a 5′-trans-splicing RNA comprising an exon and an intron, wherein the exon comprises a label sequence.

21. A method for producing 5′-labeled chimeric cDNAs, the method comprising:
a) obtaining the 5′-labeled chimeric RNAs of claim 20;
b) synthesizing one or more antisense cDNAs by incubating the RNA preparation in a mixture comprising a downstream primer and a reverse transcriptase;
c) synthesizing a 5′-labeled sense cDNA by incubating one or more of the antisense cDNAs in a mixture comprising an enzyme that catalyzes polynucleotide synthesis and an upstream primer that hybridizes to at least a portion of the exon of the 5′-trans-splicing nucleic acid.

22. A method for producing a 5′-labeled chimeric cDNA, the method comprising:
a) obtaining an RNA preparation from cells expressing a 5′-trans-splicing nucleic acid, wherein the 5′-trans-splicing nucleic acid comprises an exon and an intron;
b) synthesizing antisense cDNAs by incubating the RNA preparation, or a portion thereof, in a mixture comprising a downstream primer and a reverse transcriptase;
c) synthesizing double-stranded cDNAs by incubating the antisense cDNAs in a mixture comprising an upstream primer that hybridizes to at least a portion of the exon of the 5′-trans-splicing nucleic acid and an enzyme that mediates sense-strand synthesis;
d) synthesizing one or more 5′-labeled chimeric cDNA by incubating the double-stranded cDNAs, or copies thereof, in a mixture comprising an upstream primer that comprises a label, a downstream primer and an enzyme that mediates polynucleotide synthesis.

23. An in vitro method for producing 5′-labeled chimeric RNAs or cDNAs, the method comprising: selectively ligating an oligonucleotide to the 5′ end of capped mRNAs, wherein the oligonucleotide comprises a recognition sequence for a cleavage reagent having a cleavage site that is at least 7 base pairs distant from the recognition site.

24. The method of claim 23, wherein the oligonucleotide is selected from the group consisting of: a single-stranded RNA, a single-stranded DNA, a single-stranded DNA-RNA hybrid and a double-stranded nucleic acid.

25. The in vitro method of claim 23, wherein selectively ligating an oligonucleotide to the 5′-end of capped mRNAs in an RNA preparation comprises:
a) exposing the RNA preparation to an enzyme that catalyzes the removal of phosphate from the 5′-end of RNAs not having a 5′-cap, to obtain a first processed RNA preparation;
b) exposing the first processed RNA preparation to an enzyme that catalyzes the conversion of an RNA comprising a 5′-cap into an RNA comprising a 5′-phosphate, to obtain a second processed RNA preparation; and
c) reacting the second processed RNA preparation with a mixture comprising a ligase and the oligonucleotide.

26. The method of claim 25, wherein the ligase is a T4 RNA ligase.

27. The method of claim 25, wherein the RNA preparation is enriched for poly-A RNA.

28. The method of claim 25, wherein the enzyme that catalyzes the removal of phosphate from the 5′ end of RNAs not having a 5′-cap is calf intestinal phosphatase.

29. The method of claim 25, wherein the enzyme that catalyzes the conversion of an RNA comprising a 5′-cap into an RNA comprising a 5′-phosphate is tobacco acid pyrophosphatase.

30. The method of claim 23, wherein the oligonucleotide comprises a recognition sequence selected from the group consisting of: 5′-CTGGAG-3′,5′-GTGCAG-3′,5′-TCCGAC-3′,5′-CAGCAG-3′.

31. The method of claim 23, wherein the cleavage reagent is a restriction enzyme.

32. The method of claim 31, wherein the restriction enzyme is selected from the group consisting of: a type IIs restriction enzyme and a type III restriction enzyme.

33. The method of claim 23, further comprising synthesizing a cDNA of the ligated RNA.

34. A method for identifying sequences at or near the 5′ ends of 5′-labeled chimeric cDNA having a chimeric junction, the method comprising:
a) digesting the 5′-labeled-chimeric cDNA with a tagging cleavage reagent that cleaves at a position at least 7 base pairs in the 3′ direction from the chimeric junction, thereby releasing a 5′ portion of the cDNA;
b) selectively obtaining the 5′ portion of the cDNA;
c) sequencing at least part of the 5′ portion of the cDNA.

35. The method of claim 34, wherein the cDNA comprises an affinity purification label at or near the 5′-end, and wherein selectively obtaining the 5′ portion of the cDNA comprises contacting the cDNA with a capture medium that binds to the affinity purification label.

36. The method of claim 34, wherein sequencing at least part of the 5′ portion of the cDNAs comprises:
a) forming concatemers comprising a plurality of 5′ portions of cDNA; and
b) sequencing one or more of the concatemers.

37. The method of claim 36, wherein forming nucleic acid concatemers comprises,
i) ligating an adapter to the 3′ ends of the selectively obtained 5′ portions of cDNA, thereby making cDNA-adapters;
ii) amplifying the cDNA-adapters using a 5′ oligonucleotide primer comprising a first anchor cleavage reagent recognition site and a 3′ oligonucleotide primer comprising a second anchor cleavage reagent recognition site, thereby making amplified products that comprise a first anchor cleavage reagent recognition site and a second anchor cleavage reagent recognition site;
iii) digesting the amplified products with the first and second anchor cleavage reagents, thereby making double-digested amplified products; and
iv) ligating the double-digested amplified products to form nucleic acid concatemers.

38. The method of claim 37, wherein amplifying the cDNA-adapters using the 5′ oligonucleotide primer destroys the recognition site for the tagging cleavage reagent.

39. The method of claim 36, wherein forming nucleic acid concatemers comprises:
i) ligating one of n different adapters to the 3′ end of the selectively obtained 5′ portions of cDNA, wherein each of the n different adapters comprises a distinct second anchor cleavage reagent recognition site or the absence of a second anchor cleavage reagent recognition site, thereby making n populations of cDNA-adapters having a common first anchor cleavage reagent recognition site at or near the 5′ end having a distinct second anchor cleavage reagent recognition site or no second anchor cleavage reagent recognition site at or near the 3′ end, with the proviso that no more than two of the n different adapters have no second anchor cleavage reagent recognition site;
ii) forming nucleic acid concatemers by the iterated process of digestion with each distinct second anchor cleavage reagent and directed ligation of the digested nucleic acid ends.

40. The method of claim 39, wherein n is six, and wherein the first adapter comprises a first second anchor cleavage reagent recognition site, the second adapter comprises a second second anchor cleavage reagent recognition site, the third adapter comprises a third second anchor cleavage reagent recognition site, the fourth adapter comprises a fourth second anchor cleavage reagent recognition site, and the fifth and sixth adapters do not have a second anchor cleavage reagent recognition site.

41. The method of claim 34, wherein the cleavage reagent is a restriction enzyme.

42. The method of claim 34, wherein the 5′-labeled chimeric cDNAs are derived from 5′-chimeric RNAs from cells expressing a 5′-trans-splicing nucleic acid.

43. The method of claim 34, wherein the 5′-labeled chimeric cDNAs are derived from a population of 5′-chimeric RNAs prepared by selectively ligating an oligonucleotide to the 5′-end of capped RNAs.

44. A method for identifying, in a genome, a high probability match for a TAG sequence derived from a 5′-chimeric cDNA, the method comprising:
a) identifying one or more genomic sequences that match the TAG sequence, to obtain one or more matched genomic sequences;
b) determining whether the matched genomic sequences are located within a predicted or known transcript having the same 5′-3′ orientation as the TAG sequence;
wherein a high probability match for a TAG sequence is a matched genomic sequence that is located within a predicted or known transcript having the same 5′-3′ orientation.

45. The method of claim 44, wherein one or more of (a) and (b) is performed by a computer.

46. A computer-readable storage medium comprising instructions for performing the method of claim 44.

47. A nucleic acid construct comprising:
a) a 5′-trans-splicing nucleic acid comprising an exon and an intron; and
b) a promoter that stimulates expression of the 5′-trans-splicing nucleic acid in a cell selected from the group consisting of: a chordate cell, a protozoan cell, an arthropod cell, a fungal cell, a plant cell and a trematode cell.

48. The nucleic acid construct of claim 47, wherein the exon comprises a recognition site for a restriction enzyme that cleaves DNA at a position at least 7 base pairs removed from the recognition site.

49. The nucleic acid construct of claim 47, wherein the recognition site is for a restriction enzyme selected from the group consisting of: a type IIs restriction enzyme and a type III restriction enzyme.

50. The nucleic acid construct of claim 47, wherein the intron comprises a nucleic acid sequence that is at least 80% identical to a sequence selected from the group consisting of SEQ ID No:2 and SEQ ID No:4.

51. The nucleic acid construct of claim 47, wherein the promoter is selected from the group consisting of: a cell-specific promoter, an inducible promoter and a constitutive promoter.

52. A vector comprising the nucleic acid construct of claim 47.

53. A cell comprising the nucleic acid construct of claim 47.

54. A probe array, comprising a plurality of probes having sequences that correspond to sequences at or near the 5′-ends of a plurality of RNAs, wherein the plurality of probes distinguish between 5′ alternative transcripts encoded by the corresponding genes and wherein the plurality of probes are affixed in a spatially addressable array.

55. The probe array of claim 54, comprising at least 100 different probes having sequences that correspond to sequences at or near the 5′-ends of a plurality of RNAs.

56. A method for making a probe array comprising:
a) identifying sequences at or near the 5′-end of a plurality of RNAs
b) forming a probe array comprising probes having sequences that correspond to the sequences at or near the 5′end of a plurality of RNAs.

57. A method of claim 56, wherein the probe array comprises at least 100 probes having sequences that correspond to the sequences at or near the 5′end of a plurality of RNAs.


SEQUENCE LISTING (13 sequences)

SEQ ID NO: 1 - DNA, 22 bp, C. elegans SL-1 Exon
ggtttaattacccaagtttgag

SEQ ID NO: 2 - DNA, 83 bp, C. elegans SL-1 Intron
gtaaacattgaaactgacccaaagaaatttggcgttagctataaattttggaacgtctcctctcggggagacaaaaatactaa

SEQ ID NO: 3 - DNA, 105 bp, C. elegans SL-1 Exon/Intron
ggtttaattacccaagtttgaggtaaacattgaaactgacccaaagaaatttggcgttagctataaattttggaacgtctcctctcggggagacaaaaatactaa

SEQ ID NO: 4 - DNA, 22 bp, C. elegans SL-2 Exon
ggttttaacccagttactcaag

SEQ ID NO: 5 - DNA, 88 bp, C. elegans SL-2 Intron
gtacgctggagttctgacctttcgaaagaaagtgtcaaacgactttaatttttggaaccgctctgctggggtcatccggtagagcaaa

SEQ ID NO: 6 - DNA, 110 bp, C. elegans SL-2 Exon/Intron
ggttttaacccagttactcaaggtacgctggagttctgacctttcgaaagaaagtgtcaaacgactttaatttttggaaccgctctgctggggtcatccggtagagcaaa

SEQ ID NO: 7 - DNA, 55 bp, C. elegans mSL-1a Exon
ccttacctgtctgcctaactccttccgaggatccacggatggcggaagagtgcag

SEQ ID NO: 8 - DNA, 54 bp, C. elegans mSL-1b Exon
ccttacctgtctgcctaactccttccgaggatccacgacgggcggaagtttgag

SEQ ID NO: 9 - DNA, 60 bp, Artificial Sequence
  Concatemer of experimentally-derived sequence at or near the 5′-end of an acceptor C. elegans SL-1 mRNA
ctgcaggtaaacattgaaacggccgtttcaatgtttacctgcaggtaaacattgaaacgg

SEQ ID NO: 10 - DNA, 16 bp, Artificial Sequence
  Sense strand of experimentally-derived sequence at or near the 5′-end of an acceptor C. elegans SL-1 mRNA containing the AG sequence
agaatgaagactcttc

SEQ ID NO: 11 - DNA, 16 bp, Artificial Sequence
  Antisense strand of experimentally-derived sequence at or near the 5′-end of an acceptor C. elegans SL-1 mRNA containing the AG sequence
gaagagtcttcattct

SEQ ID NO: 12 - DNA, 66 bp, C. elegans genomic sequence
ttttgatgcttcagtgtttcatttcaatttattttattacagaatgaagactcttcttgtactagc

SEQ ID NO: 13 - DNA, 36 bp, Artificial Sequence
  Modified SL-1 exon sequence fused to a portion of C. elegans gpd-2 mRNA
ctttggcatggctcaaacttccgcccgtcgtggatc